(19) United States
(12) Patent Application Publication
(10) Pub. No.: US 2025/0228850 A1
(43) Pub. Date: Jul. 17, 2025

(54) COMBINATION OF A CXCR7 ANTAGONIST WITH AN S1P1 RECEPTOR MODULATOR

(71) Applicant: Idorsia Pharmaceuticals Ltd., Allschwil (CH)

(72) Inventor: Laetitia POUZOL, Allschwil (CH)

(21) Appl. No.: 17/773,216

(22) PCT Filed: Oct. 30, 2020
(86) PCT No.: PCT/EP2020/080510

§ 371 Date: Aug. 2, 2022

(30) Foreign Application Priority Data
Oct. 31, 2019 (WO) PCT/EP2019/079932

(51) Int. Cl. A61K 31/506; A61K 31/137; A61K 31/397; A61K 31/404; A61K 31/4245; A61K 31/426; A61K 31/4439; A61P 25/00; A61P 25/28; A61P 29/00; A61P 37/06

(52) U.S. Cl. CPC A61K 31/506; A61K 31/137; A61K 31/397; A61K 31/404; A61K 31/4245; A61K 31/426; A61K 31/4439; A61P 25/00; A61P 25/28; A61P 29/00; A61P 37/06

(57) ABSTRACT

The present invention relates to the compound (3S,4S)-1-Cyclopropylmethyl-4-{[5-(2,4-difluoro-phenyl)-isoxazole-3-carbonyl]-amino}-piperidine-3-carboxylic acid (1-pyrimidin-2-yl-cyclopropyl)-amide: (Formula) and its use as modulator of the CXCL11/CXCL12 receptor CXCR7, especially in combination with other active ingredients or therapeutic agents comprising a sphingosine-1-phosphate receptor 1 modulator (S1P1 receptor modulator) in the prevention or treatment of diseases and disorders where both CXCR7 expression or its ligands and S1P play a role. The invention further relates to pharmaceutical compositions comprising COMPOUND in combination with said other active ingredient(s) or therapeutic agent(s).

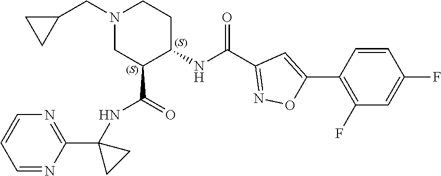

The present invention concerns the compound (3S,4S)-1-Cyclopropylmethyl-4-{[5-(2,4-difluoro-phenyl)-isoxazole-3-carbonyl]-amino}-piperidine-3-carboxylic acid (1-pyrimidin-2-yl-cyclopropyl)-amide (hereinafter also referred to as “COMPOUND”):

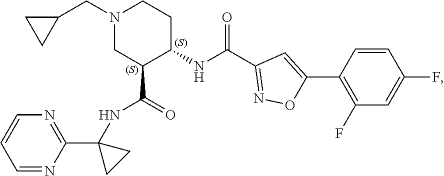

and its use as modulator of the CXCL11/CXCL12 receptor CXCR7, in combination with other active ingredients or therapeutic agents comprising a sphingosine-1-phosphate receptor 1 modulator (S1P1 receptor modulator) in the prophylaxis/prevention or treatment of diseases and disorders where both CXCR7 expression or its ligands and S1P play a role. The invention further relates to pharmaceutical compositions comprising COMPOUND in combination with said other active ingredient(s) or therapeutic agent(s). The invention further relates to daily doses of COMPOUND that, when administered for example once or twice daily, may be well tolerated and pharmaceutically effective in the prophylaxis/prevention or treatment of diseases and disorders where CXCR7 expression or its ligands play a role.

COMPOUND is known as a modulator of the CXCL11/CXCL12 receptor CXCR7 from WO2018/019929. A crystalline form of COMPOUND is known from WO2019/145460. COMPOUND may be of potential use in the prophylaxis/prevention or treatment of certain diseases and disorders relating to the CXCR7 receptor or its ligands including:
cancer such as brain tumors including malignant gliomas, glioblastoma multiforme; neuroblastoma; pancreatic cancer including pancreatic adenocarcinoma/pancreatic ductal adenocarcinoma; gastro-intestinal cancers including colon carcinoma, hepatocellular carcinoma and gastric cancer; Kaposi's sarcoma; leukemias including adult T-cell leukemia; lymphoma; lung cancer; breast cancer; rhabdomyosarcoma; prostate cancer; esophageal squamous cancer; oral squamous cell carcinoma; endometrial cancer; thyroid carcinoma including papillary thyroid carcinoma; metastatic cancers; lung metastasis; skin cancer including melanoma and metastatic melanoma; bladder cancer; multiple myelomas; osteosarcoma; head and neck cancer; and renal carcinomas including renal clear cell carcinoma, metastatic renal clear cell carcinoma;
inflammatory diseases such as chronic rhinosinusitis, asthma, chronic obstructive pulmonary disorder, atherosclerosis, myocarditis, acute lung injury, endometriosis, uveitis, diabetic retinopathy and sarcoidosis;
autoimmune disorders such as (inflammatory) demyelinating diseases; multiple sclerosis (MS); Guillain Barre syndrome; rheumatoid arthritis (RA); inflammatory bowel diseases (IBD, especially comprising Crohn's disease and ulcerative colitis); systemic lupus erythematosus (SLE) including neuropsychiatric systemic lupus erythematosus and lupus nephritis; interstitial cystitis; celiac disease; autoimmune encephalomyelitis; osteoarthritis; type I diabetes; psoriasis; autoimmune thyroiditis; Sjögrens Syndrome; ankylosing spondylitis and vitiligo;
neurodegenerative disorders such as amyotrophic lateral sclerosis;
transplant rejection (notably renal allograft rejection, cardiac allograft rejection, and graft-versus-host diseases brought about by hematopoietic stem cell transplantation);
fibrosis (notably liver fibrosis, liver cirrhosis, lung fibrosis, cardiac fibrosis, especially idiopathic pulmonary fibrosis; and
ischemic injury such as renal ischemia or cerebral ischemia.

Chemokine receptors are a group of G-protein coupled receptors (GPCRs) that bind peptidic chemokine ligands with high affinity. The predominant function of chemokine receptors is to guide leukocyte trafficking to lymphoid organs and tissues under resting conditions as well as during inflammation, but a role for certain chemokine receptors on non-hematopoietic cells and their progenitors has also been recognized.

CXCR7 (alias ACKR3, alias RDC1, alias CMKOR1, alias GPR159) has two known chemokine ligands: CXCL12 (alias stromal cell-derived factor 1, SDF-1; alias Pre-B cell growth stimulating factor, PBSF) and CXCL11 (alias I-TAC, alias IFN-g-inducible T cell chemo-attractant). Binding of either CXCL11 or CXCL12 to CXCR7 leads to internalization of the CXCR7-ligand complex (Burns J M et al. J Exp Med 2006, 203(9):2201-13) and degradation of the ligand (Naumann U et al. PLoS One 2010, 5(2):e9175). This scavenging activity contributes to the establishment and maintenance of CXCL11 and CXCL12 concentration gradients from blood vessels toward tissues.

CXCL12, a stroma-derived chemo-attractant, participates in the immune surveillance and in the regulation of inflammatory responses. CXCL12 is secreted by bone marrow stromal cells, endothelial cells, heart, skeletal muscle, liver, brain, kidney, thymus, lymph nodes, parenchymal cells and plays an essential role in stem cell proliferation, survival, and homing of hematopoietic/progenitor to the bone marrow (Rankin S M et al.; Immunol let. 2012, 145(1-2):47-54). CXCL12 is induced under certain pathological disorders including ischemia, inflammation, hypoxia, cancer, neurodegenerative diseases and autoimmune diseases (Juarez J et al. Curr Pharm Des 2004, 10(11):1245-59).

CXCL12 also recruits bone-marrow derived progenitor cells to sites of vasculature formation. Moreover, it plays a prominent role in carcinogenesis. CXCL12 promotes the recruitment of endothelial progenitor cells and of myeloid derived suppressor cells to the tumor sites as well as other bone marrow derived cells. CXCL12 also plays a role during inflammation acting on cell migration, adhesion and survival (Kumar R et al. Cell Immunol. 2012, 272(2):230-41). CXCL12 also drives differentiation, maturation of cells such as oligodendrocyte progenitors (Gottle P et al. Ann Neurol. 2010, 68(6):915-24).

CXCL11 is mainly expressed in the pancreas, peripheral blood leukocytes, thymus, liver, spleen, and lung. This chemokine is induced by interferon and is up-regulated during infection or cancer process (Cole et al. J Exp Med. 1998, 187(12):2009-21).

In addition to CXCR7, CXCL12 binds and activates CXCR4 (alias Fusin, alias Leukocyte-derived seven-transmembrane-domain receptor; LESTR, alias D2S201E, alias seven-transmembrane-segment receptor, alias HM89, alias lipopolysaccharide-associated protein 3; lap3, alias LPS-associated protein 3) while CXCL11 binds and activate CXCR3 (alias GPR9, alias CD183).

The interaction of CXCR7 and its ligands CXCL12 and CXCL11 (henceforth referred to as the CXCR7 axis) is thus involved in guiding receptor bearing cells to specific locations in the body, particularly to sites of inflammation, immune injury and immune dysfunction and is also associated with tissue damage, the induction of apoptosis, cell growth and angiostasis. CXCR7 and its ligands are upregulated and highly expressed in diverse pathological situations including cancer, autoimmune disorders, inflammation, infection, transplant rejection, fibrosis and neurodegeneration.

CXCR7 modulators have been disclosed to be of potential use, alone, or in combination, in diseases where CXCR7 modulation (e.g. using siRNA, shRNA, microRNAs, overexpression, CXCR7 knock-out animals, CXCR7 agonists, CXCR7 antagonists, antibodies or nanobodies) has been shown to regulate leukocyte migration (Berahovich R D et al.; Immunology. 2014, 141(1):111-22) and to promote myelin/neuronal repair (Williams J L et al.; J Exp Med. 2014, 5; 211(5):791-9; Gottle P et al.; Ann Neurol. 2010, 68(6):915-24), providing beneficial effects in experimental disease models of inflammatory, autoimmune and demyelinating diseases, including multiple sclerosis and autoimmune encephalomyelitis (Cruz-Orengo L et al.; J Neuroinflammation. 2011, 6; 8:170; Bao J et al.; Biochem Biophys Res Commun. 2016 Jan. 1; 469(1):1-7), Guillain-Barre syndrome or autoimmune neuritis (Brunn A et al.; Neuropathol Appl Neurobiol. 2013, 39(7):772-87), and rheumatoid arthritis (Watanabe K et al.; Arthritis Rheum. 2010, 62(11):3211-20).

Specifically, the impact of CXCR7 on inflammatory demyelinating diseases is known from the literature. CXCR7 is expressed in various regions throughout the adult mouse brain and its expression is upregulated in mouse model for multiple sclerosis (MS) and during demyelination in a non-inflammatory demyelination model (Banisadr G et al.; J Neuroimmune Pharmacol. 2016 March; 11(1):26-35; Williams J L et al.; J Exp Med. 2014, 5; 211(5):791-9; Gottle P et al.; Ann Neurol. 2010, 68(6):915-24). Altered expression patterns of CXCL12 at the blood-brain barrier (BBB) is involved in multiple sclerosis and correlates with severity of the disease (McCandless E E et al.; Am J Pathol. 2008, 172(3):799-808). CXCR7 functional antagonism has been shown to be effective in experimental autoimmune encephalomyelitis in mice. Those recent studies strongly implicate CXCR7 as a disease-modifying molecule in multiple sclerosis via complementary mechanisms: (i) by facilitating leukocyte entry into the perivascular space via CXCL12 redistribution at the BBB (Cruz-Orengo L et al.; J Neuroinflammation. 2011, 6; 8:170; Cruz-Orengo L et al.; J Exp Med. 2011, 14; 208(2):327-39) and regulating the CXCR4-mediated activation of integrins (Hartmann T N et al.; J Leukoc Biol. 2008; 84(4):1130-40) (ii) by direct effect on microglial chemotaxis (Bao J et al.; Biochem Biophys Res Commun. 2016 Jan. 1; 469(1):1-7) and on inflammatory monocytes, facilitating their entry into the brain (Douglas S D et al.; J Leukoc Biol. 2017; 102: 1155-1157) (iii) by promoting remyelination via increased levels of CXCL12 enhancing CXCR4-mediated oligodendrocyte progenitor cell maturation (Williams J L et al.; J Exp Med. 2014, 5; 211(5):791-9; Gottle P et al.; Ann Neurol. 2010, 68(6):915-24). Recently, Chu et al (Neuroscientist. 2017, 23(6): 627-648) reviewed the importance of targeting the CXCL12/CXCR4/CXCR7 axis for demyelinating diseases, due to their central role in promoting the migration, proliferation and differentiation of oligodendrocyte progenitor cells. Thus, CXCR7 antagonism could therapeutically prevent inflammation and enhance myelin repair in the demyelinated adult CNS.

Specifically, the potential role of CXCR7 in rheumatoid arthritis is known from the literature. CXCR7 is reported to be expressed on endothelial cells in the synovium. Also, elevated levels of CXCL12 and CXCL11 mRNA were found in synovial tissue of rheumatoid arthritis patients (Ueno et al.; Rheumatol Int. 2005, 25(5):361-7).

CXCL12 was shown to play a central role in CD4⁺ T cell and monocytes accumulation in the synovium (Nanki T et al.; J Immunol. 2000, 165(11):6590-8; Blades M C et al.; Arthritis Rheum. 2002 March; 46(3):824-36). In addition, CXCL12 participates in the rheumatoid arthritis process via its pro-angiogenic functions and its action on osteoclast recruitment and differentiation. Therefore, modulators of the CXCL12 pathway including CXCR7 modulators have been proposed as potential therapeutic agents to treat rheumatoid arthritis. Villalvilla et al (Expert Opin Ther Targets. 2014, 18(9):1077-87) recently discussed preclinical and clinical data that support the potential use of anti-CXCL12 agents in rheumatoid arthritis treatments. Watanabe et al (Arthritis Rheum. 2010, 62(11):3211-20) demonstrated that a CXCR7 inhibitor prophylactically and therapeutically reduced disease clinical signs and angiogenesis in a mouse collagen-induced arthritis model.

CXCR7 is further reported to be involved in several inflammatory disorders including acute and chronic lung inflammatory processes such as chronic obstructive pulmonary disease, acute lung injury, asthma, pulmonary inflammation, lung fibrosis, as well as atherosclerosis, liver fibrosis, and cardiac fibrosis.

CXCL12 and CXCL11 are also reported to be upregulated in inflammatory bowel diseases (Koelink P J et al.; Pharmacol Ther. 2012, 133(1):1-18). CXCR7 was found upregulated on peripheral blood T cells in Inflammatory Bowel Diseases (IBD) (Werner L et al.; J Leukoc Biol. 2011, 90(3):583-90). The author hypothetise that “the increased expression of CXCR7 in the peripheral blood of IBD patients could foster increased influx of T cells to sites of mucosal inflammation” (Werner L et al.; Theranostics. 2013, 3(1):40-6). In mouse models for IBD, modulators of the CXCL12 pathway could decrease infiltration of T cells and reduce tissue damage (Mikami S et al.; J Pharmacol Exp Ther. 2008, 327(2):383-92; Xia X M et al.; PLoS One. 2011, 6(11):e27282).

Elevated levels of CXCL12 and CXCL11 have also been found in lesional psoriatic skin (Chen S C et al.; Arch Dermatol Res. 2010, 302(2):113-23; Zgraggen S et al.; PLoS One. 2014, 9(4):e93665). Zgraggen et al demonstrated that blockade of CXCL12 improved the course of chronic skin inflammation in two different models of psoriasis-like skin inflammation.

Several other auto-immune disorders like systemic lupus erythematosus (SLE) display altered CXCR7/CXCR4 expression correlated with an impaired CXCL12-promoted migration of SLE B cells (Biajoux V et al.; J Transl Med. 2012, 18; 10:251). In addition, CXCL12 was significantly up-regulated in the nephritic kidneys in multiple murine models of lupus. Wang et al. (J Immunol. 2009, 182(7):4448-58) showed that acting on the CXCL12 axis is a good therapeutic target in lupus, as a CXCR4 antagonist significantly ameliorated the disease, prolonging survival and reducing nephritis and lymphoproliferation.

Matin et al (Immunology. 2002, 107(2):222-32) demonstrated that blockade of CXCL12 with antibodies resulted in reduction of diabetes development and inhibition of insulitis in a mouse model of diabetes.

CXCL12 and CXCR4 were found upregulated in thyroids from autoimmune patients and in animal models (Armengol M P et al.; J Immunol. 2003, 170(12):6320-8). Liu et al. (Mol Med Rep. 2016, 13(4):3604-12) disclose that blocking of CXCR4 reduced the severity of autoimmune thyroiditis in mice, decreasing the lymphocytes infiltration and autoantibodies production.

CXCR4 was found upregulated in synovial tissues from patients with ankylosing spondylitis (He C et al.; Mol Med Rep. 2019, 19(4):3237-3246). CXCR4 inhibition resulted in reduced fibroblast proliferation and osteogenesis.

Neurodegenerative disorders have been shown to display altered CXCL12/CXCR4 expression. This pathway is involved in recruitment and differentiation of self-renewing and multipotent neural progenitor cells which play a critical role during tissue repair. Meizhang et al reviewed the role of CXCL12 in neurodegenerative diseases and the impact of manipulations of the CXCL12 signaling pathway on neurodegenerative disorders in animal models (Meizhang et al. Trends Neurosci. 2012, 35(10): 619-628). Recently, the expression of CXCL12 and CXCR4 were found upregulated in peripheral blood of Parkinson's disease patients (Bagheri et al. Neuroimmunomodulation. 2018, 25(4):201-205). CXCR4/CXCL12 pathway has also been involved in the inflammatory process taking place in Alzheimer's disease (Hongyan et al. Brain Circ. 2017, 3(4):199-203). Rabinovich-Nikitin et al, teach that blocking the CXCR4/CXCL12 signaling reduced microglial inflammation, blood brain barrier permeability and increased number of motor neurons, increasing survival of mice in a model of amyotrophic lateral sclerosis (ALS) (Rabinovich-Nikitin et al. J Neuroinflammation. 2016, 13: 123).

CXCR7 is also known as a scavenger receptor for several opioid peptides, especially enkephalins and dynorphins, regulating their availability and thereby the signalling through their classical opioid receptors (Meyrath M et al. Nat Commun. 2020; 11(1):3033). As CXCR7 acts as a broad-spectrum scavenger for opioid peptides, administration of a CXCR7 antagonist may lead to an increase in these opioid peptides in analogy to the increase observed for the chemokine ligands CXCL11 and CXCL12. As such, modulation of endogenous opioid levels may be of use in clinical pain management and control of nociception (Holden J E et al. AACN Clin Issues. 2005; 16(3): 291-301). Cerebrospinal fluid levels of prodynorphin-derived peptides, which have been shown to bind to CXCR7, are reduced in Huntington's disease patients (Al Shweiki M R et al. Mov Disord. 2020; doi: 10.1002/mds.28300), hence increasing the levels of these peptides by administration of a CXCR7 antagonist may be beneficial in this disease. Endogenous opioid peptides are also implicated in mood disorders, such as depression (Peciña M et al. Mol Psychiatry. 2019; 24(4): 576-587). It can therefore be expected that modulating endogenous peptide levels by blocking the scavenging receptor CXCR7 may be used to treat mood disorders. Along those lines, a CXCR7 modulator has been shown to have anxiolytic activity (Ikeda Y et al. Cell. 2013; 155(6): 1323-36) in a preclinical model. Therefore, CXCR7 modulators may, in addition to the diseases and disorders relating to the CXCR7 receptor or its ligands mentioned above, also be useful for the prophylaxis/prevention or treatment of certain diseases and disorders relating to opioid receptor signalling, including neuropathic pain, neurodegenerative diseases including Huntington's disease, addiction disorders, mood disorders, anxiety disorders.

Several sphingosine-1-phosphate receptor 1 modulators (alternatively named S1P1 receptor modulators, comprising non-selective S1P1 receptor modulators such as fingolimod, as well as selective S1P1 receptor modulators) are S1P1 receptor agonists which act pharmacologically as functional antagonists on the S1P1 receptor. S1P1 receptor modulators have been described as being useful for the prevention and/or treatment of diseases or disorders associated with an activated immune system (Juif et al., Exp. Op. Drug Metabol. & Tox. (2016) 12(8), 879-895). S1P1 receptor modulators indirectly antagonize the S1P1 receptor's function and sequester lymphocytes in lymph nodes (Subei et al, CNS Drugs. 2015 July; 29(7): 565-575). It was confirmed that multiple S1P1 receptor modulators signal in an identical manner through S1P1, leading to S1P1 receptor degradation (Lukas et al., J. Biomol. Screening (2014) 19(3) 407-416). In clinical practice, S1P1 receptor modulators including non-selective and selective S1P1 receptor modulators show a risk for bradyarrhythmia and atrioventricular blocks (AV blocks). In consequence, for example for fingolimod it is recommended that heart rate and blood pressure should generally be monitored during treatment initiation in patients. Mitigation of risk by using up-titration dosage regimens have been proposed and such dosage regimens are used in clinical practice (see for example for fingolimod: WO2006/058316, WO2010/075239, WO2011/041145, WO2013/055833; for ponesimod: WO2009/115954, WO2016/091996; for siponimod: WO2010/072703, WO2013/057212, WO2015/155709). Another potential caveat of certain S1P1 receptor modulators available or in development is that treatment generally leads to lymphopenia, and may in certain cases lead to severe lymphopenia, associated with an increased risk of infection. A combination with another active principle could result in increased efficacy and/or the presence of higher lymphocyte counts. Furthermore, combination with another active principle could allow for a reduction of the minimal efficacious dose of an S1P1 receptor modulator. Thus, such combination with another active principle may be of advantage, especially in case the immune system may need to be re-activated for example in an emergency situation such as an acute infection. S1P1 receptor modulators have in particular been described as possessing a unique mechanism of action in the treatment of multiple sclerosis (MS) (Chaudhry et al. Neurotherapeutics (2017) 14:859-873). MS is a chronic inflammatory and demyelinating disease of the CNS, in which the inflammatory process is associated with a destruction of myelin, leading to the appearance of large focal lesions of demyelination. Axonal damage and loss as consequences of the inflammatory demyelination also occur, even if at variable extents. Active remyelination processes can at least in part repair myelin lesions, whereas axonal loss is permanent and irreversible. MS is primarily considered an autoimmune neurodegenerative disease, that is, a disease caused by an adaptive immune response to self-antigens. In MS, activated myelin-reactive T cells are recruited from the periphery to the CNS, leading to the activation of microglia and to the recruitment of circulating macrophages (Grassi et al. Frontiers in Pharmacology 2019, doi:10.3389/fphar.2019.00807).

S1P1 receptor modulators have been disclosed to be of potential use in neuro-degenerative diseases where S1P1 receptor modulators have a direct effect on CNS resident cells such as microglia, astrocytes, neurons, oligodendrocyte progenitor cells and oligodendrocytes (Miron et al. J Neurol Sci. 2008, 274(1-2):13-7), providing beneficial effects in experimental disease models of neurodegeneration.

Specifically, the impact of S1P on neurodegenerative diseases is known from the literature. Yazdi et al. recently discussed the experimental and clinical studies that support direct effects of a S1P1 receptor modulator on myelination (Yazdi et al. J Neuro Res. 2019, 00:1-13). Angelopoulou et al. recently reviewed the involvement of S1P in Alzheimer's disease (AD) pathogenesis and the beneficial effects of a S1P1 receptor modulator in AD models (Angelopoulou et al. Neuromolecular Med. 2019, 21(3):227-238). A S1P1 receptor modulator was shown to be able to reduce neurological deficits and to extend the survival of mice in a model of amyotrophic lateral sclerosis (ALS), modulating the neuroinflammatory response and increasing expression of brain-derived neurotrophic factor (Potenza et al. Neurotherapeutics. 2016, 13(4): 918-927). Miguez et al. teach that a S1P1 receptor modulator ameliorates hippocampal synaptic plasticity and memory in a mouse model of Huntington's disease, reducing astrogliosis and decreasing local inflammation (Miguez et al. Hum Mol Genet. 2015, 24(17):4958-70).

Fingolimod (2-amino-2-[2-(4-octylphenyl) ethyl]-propane-1,3-diol, CAS Reg. No. 162359-55-9, e.g. WO2008/000419, WO2010/055027, WO2010/055028, WO2010/072703) is a non-selective S1P1 receptor modulator indicated for the treatment of the relapsing form of multiple sclerosis (MS). Fingolimod 0.5 mg once-daily is the first oral therapy approved for relapsing multiple sclerosis in many countries and for highly active relapsing-remitting MS (RRMS) in the European Union. In the U.S., fingolimod is indicated for the treatment of relapsing forms of multiple sclerosis (MS), to include clinically isolated syndrome, relapsing-remitting disease, and active secondary progressive disease, in patients 10 years of age and older with a recommended dosage for adults and pediatric patients weighing more than 40 kg of 0.5 mg orally once-daily. Fingolimod remains in the blood and has pharmacodynamic effects, including decreased lymphocyte counts, for up to 2 months following the last dose of fingolimod. Lymphocyte counts generally return to the normal range within 1-2 months of stopping therapy. Despite the long wash-out period observed, it has also been shown that reducing the dose of fingolimod, for example to 0.5 mg every other day, may lead to disease reactivation in a significant proportion of patients (Zecca et al., Multiple Sclerosis Journal (2017) 24(2), 167-174).

Ponesimod [(R)-5-[3-chloro-4-(2,3-dihydroxy-propoxy)-benz[Z]ylidene]-2-([Z]-propylimino)-3-o-tolyl-thiazolidin-4-one, CAS Reg. No. 854107-55-4, e.g. WO2005/054215, WO2008/062376, WO2010/046835, WO2014/027330] is a selective S1P1 receptor agonist and oral administration thereof results in a consistent, sustained, and dose-dependent reduction in the number of peripheral blood lymphocytes. Ponesimod has been described to be useful in the treatment and/or prevention of diseases or disorders associated with an activated immune system (see e.g. WO 2005/054215 and WO 2009/115954). In particular ponesimod has shown clinical benefit in phase II/phase III trials in patients with moderate to severe chronic plaque psoriasis and in patients with relapsing-remitting multiple sclerosis. Ponesimod may be prepared according to procedures disclosed in WO 2005/054215, WO 2008/062376 and WO 2014/027330.

Cenerimod [(S)-3-[[4-[5-(2-Cyclopentyl-6-methoxypyridin-4-yl)[1,2,4]oxadiazol-3-yl]-2-ethyl-6-methylphenyl]-oxy]-propane-1,2-diol, CAS Reg. No. 1262414-04-9, e.g. WO2011/007324, WO2013/175397, WO2016/184939, Piali et al., Pharmacol Res Perspect. 2017; e00370] is a selective S1P1 receptor agonist and entered a multiple-dose efficacy and safety study for the treatment of systemic lupus erythematosus. No up-titration dosage regimen seems to be required for cenerimod.

Siponimod (1-(4-[1-[(E)-4-Cyclohexyl-3-trifluoromethyl-benzyloxyimino]-ethyl]-2-ethyl-benzyl)-azetidine-3-carboxylic acid, CAS Reg. No. 1230487-00-9, e.g. WO2004/103306, WO2010/071794, WO2010/080409, WO2010/080455, WO2019/064184) is an S1P1 receptor modulator and was studied for the treatment of secondary progressive multiple sclerosis (SPMS), which is the progressive neurological decline of multiple sclerosis that happens independent of acute relapses. In active SPMS, siponimod decreases the risk of disability and MS relapses. In the U.S., siponimod is indicated for the treatment of relapsing forms of multiple sclerosis (MS), to include clinically isolated syndrome (defined as a first episode of neurologic symptoms that lasts at least 24 h and is caused by inflammation or demyelination in the central nervous system), relapsing-remitting disease, and active secondary progressive disease (SPMS), in adults with a recommended maintenance dosage of 2 mg orally once-daily. After stopping siponimod therapy, siponimod remains in the blood for up to 10 days. Starting other therapies during this interval will result in concomitant exposure to siponimod. Lymphocyte counts returned to the normal range in 90% of patients within 10 days of stopping therapy. However, residual pharmacodynamics effects, such as lowering effects on peripheral lymphocyte count, may persist for up to 3-4 weeks after the last dose. Use of immunosuppressants within this period may lead to an additive effect on the immune system, and therefore caution should be applied 3-4 weeks after the last dose of siponimod.

Ozanimod (5-[3-[(1S)-2,3-Dihydro-1-[(2-hydroxyethyl)amino]-1H-inden-4-yl]-1,2,4-oxadiazol-5-yl]-2-(1-methylethoxy)-benzonitrile, CAS Reg. No. 1306760-87-1, e.g. WO2011/060392, WO2015/066515, WO2018/184185, WO2018/208855, WO2018/215807, WO2019/058290, WO2019/094409) is an investigational S1P1 receptor modulator that was tested in phase III clinical trials for the therapy of relapsing forms of multiple sclerosis (RMS) (NCT02047734); and is further tested in Crohn's disease and ulcerative colitis (UC). Since 2020, ozanimod is indicated in the U.S. for the treatment of relapsing forms of multiple sclerosis (MS), to include clinically isolated syndrome, relapsing-remitting disease, and active secondary progressive disease, in adults; and in Europe for the treatment of adult patients with relapsing remitting multiple sclerosis (RRMS) with active disease as defined by clinical or imaging features.

Etrasimod [(3R)-7-[[4-Cyclopentyl-3-(trifluoromethyl)phenyl]methoxy]-1,2,3,4-tetrahydrocyclopent[b]indole-3-acetic acid, CAS Reg. No. 1206123-37-6, e.g. WO2010/011316, WO2011/094008, WO2016/112075, WO2016/209809, Al-Shamma et al, J Pharmacol Exp Ther (2019) 369:311-317] is an investigational S1P1 receptor modulator that is currently in development for example for the therapy of inflammatory bowel diseases including Crohn's disease and ulcerative colitis (UC).

Further S1P1 receptor modulators have been described and clinically tested, however, their development may have been discontinued:
Amiselimod (MT-1303, 2-Amino-2-[2-[4-(heptyloxy)-3-(trifluoromethyl)phenyl]ethyl]-1,3-propanediol, CAS Reg. No. 942399-20-4, e.g. WO2007/069712, WO2018/021517; Harada et al., Br J Clin Pharmacol (2017) 83 1011-1027; Sugahara et al., Br. J. Pharmacol. (2017) 174 15-27);
Ceralifimod (1-[[3,4-dihydro-6-[(2-methoxy-4-propylphenyl)methoxy]-1-methyl-2-naphthalenyl]methyl]-3-azetidinecarboxylic acid, CAS Reg. No. 891859-12-4, e.g. WO2006/064757, Kurata et al. JMedChem 60(23) (2017), 9508-9530);
GSK 2018682 (4-[5-[5-Chloro-6-(1-methylethoxy)-3-pyridinyl]-1,2,4-oxadiazol-3-yl]-1H-indole-1-butanoic acid, e.g. WO2008/074821);
CS-0777 (1-[5-[(3R)-3-Amino-4-hydroxy-3-methylbutyl]-1-methyl-1H-pyrrol-2-yl]-4-(4-methylphenyl)-1-butanone, CAS Reg. No. 827344-05-8, e.g. WO2005/079788, Nishi et al., Med Chem Lett. 2011 2; 2(5):368-72); and
Mocravimod (2-amino-2-[2-(2-chloro-4-{[3-(phenylmethoxy)phenyl]sulfanyl}phenyl)ethyl]propane-1,3-diol; KEP203, CAS Reg. No. 509092-16-4, e.g. U.S. Pat. Nos. 9,920,005, 6,960,692), disclosed to enter studies in high-risk acute myeloid leukemia.

It has now been found that COMPOUND, a CXCR7 antagonist having potential in the prophylaxis/prevention and treatment of diseases and disorders which respond to the activation of the CXCL12 receptors and/or CXCL11 receptors, may have complementary, and even synergistic effect when combined with S1P1 receptor modulators in the treatment of such diseases and disorders having a component of an inflammatory autoimmune response, and/or a component of a neurodegenerative response. Such combination may, thus, especially be useful in the prophylaxis/prevention and/or treatment of autoimmune and inflammatory diseases and disorders, transplant rejection, and neurodegenerative diseases and disorders (especially autoimmune diseases and disorders which have an inflammatory component, in particular autoimmune and/or inflammatory demyelinating diseases and disorders including multiple sclerosis). Additionally, a potential remyelinating pharmacological effect of COMPOUND may complement S1P1 receptor modulators which are clinically established treatment options for such inflammatory demyelinating diseases. Furthermore, the combination of COMPOUND with an S1P1 receptor modulator may allow for a dose reduction of the corresponding S1P1 receptor modulator, potentially even to dosages that are below established optimally efficacious dosages of such S1P1 receptor modulator when administered alone, thus, potentially mitigating certain safety liabilities [for example the effect on the cardiovascular system (bradycardia), and/or the long residual exposure after treatment discontinuation in a situation where exposure to an S1P1 receptor modulator is contraindicated, and/or the (potentially severe) lymphopenia] known to be associated with certain S1P1 receptor modulators.

### DESCRIPTION OF THE FIGURES

FIG. 1 shows the dose-dependent effect of COMPOUND on the overall extent of the EAE disease as assessed by cumulative disease scores.

FIG. 2 shows the dose-dependent effect of COMPOUND on CXCL12 plasma concentration in the mouse MOG-induced EAE model.

FIG. 3 shows the effect of fingolimod (0.03 mg/kg, q.d.) on the overall extent of EAE disease as assessed by cumulative disease scores.

FIG. 4 shows the therapeutic efficacy of COMPOUND, fingolimod, and their combination on average clinical score in EAE mouse model.

FIG. 5 shows the therapeutic effect of COMPOUND, fingolimod, and their combination on severity of the mouse EAE disease, represented as the maximal clinical score.

FIG. 6 shows the therapeutic effect of COMPOUND, fingolimod, and their combination on neurofilament light chain plasma concentration in a mouse EAE model.

FIG. 7 shows the effect of COMPOUND, fingolimod, and their combination on blood lymphocyte count in a mouse EAE model.

FIG. 8 shows the effect of COMPOUND, fingolimod, and their combination on plasma CXCL12 concentrations in a mouse EAE model.

FIG. 9 shows the direct effects of COMPOUND on myelination as determined in a cuprizone-induced demyelination mouse model.

FIG. 10 shows the effects of COMPOUND on mature oligodendrocyte numbers in the mouse cuprizone-induced demyelination model.

FIG. 11 shows the therapeutic effect of COMPOUND or fingolimod starting one week before cuprizone withdrawal on demyelination/remyelination in the mouse cuprizone-induced demyelination model.

FIG. 12 shows the dose-dependent effect of COMPOUND on the overall extent of EAE disease as assessed by cumulative disease scores.

FIG. 13 shows the dose-dependent effect of COMPOUND on plasma CXCL12 concentrations in the mouse PLP-induced EAE model.

FIG. 14 shows the dose-response relationship of peak CXCL12 plasma concentrations after single dose in human healthy subjects.

FIG. 15 shows the predicted exposure response relationship at steady-state stratified by dose.

### DETAILED DESCRIPTION OF THE INVENTION

1) A first embodiment relates to a pharmaceutical composition comprising, as active principles, COMPOUND, or a pharmaceutically acceptable salt thereof, in combination with an S1P1 receptor modulator, or a pharmaceutically acceptable salt thereof, as well as at least one pharmaceutically acceptable (inert) excipient.

The pharmaceutical composition according to embodiment 1) can be used as medicament, e.g. in the form of pharmaceutical compositions for enteral (such especially oral) or parenteral administration (including topical application or inhalation).

2) A further embodiment relates to a pharmaceutical composition according to embodiment 1), wherein the S1P1 receptor modulator is fingolimod, ponesimod, siponimod, ozanimod, cenerimod, etrasimod, amiselimod, ceralifimod, GSK 2018682, or CS-0777; or, in addition, mocravimod (especially fingolimod, ponesimod, siponimod, or ozanimod; or, in addition, cenerimod); or a pharmaceutically acceptable salt thereof. 3) A further embodiment relates to a pharmaceutical composition according to embodiment 1), wherein the S1P1 receptor modulator, or a pharmaceutically acceptable salt thereof, is fingolimod, ponesimod, siponimod, ozanimod, cenerimod, or etrasimod (especially fingolimod, ponesimod, siponimod, or ozanimod), or a pharmaceutically acceptable salt thereof.

4) A further embodiment relates to a pharmaceutical composition according to embodiment 1), wherein the S1P1 receptor modulator, or a pharmaceutically acceptable salt thereof, is cenerimod, etrasimod, or amiselimod (especially cenerimod or etrasimod), or a pharmaceutically acceptable salt thereof.

5) A further embodiment relates to a pharmaceutical composition according to embodiment 1), wherein the S1P1 receptor modulator, or a pharmaceutically acceptable salt thereof, is fingolimod, or a pharmaceutically acceptable salt thereof.

6) A further embodiment relates to a pharmaceutical composition according to embodiment 1), wherein the S1P1 receptor modulator, or a pharmaceutically acceptable salt thereof, is ponesimod, or a pharmaceutically acceptable salt thereof.

7) A further embodiment relates to a pharmaceutical composition according to embodiment 1), wherein the S1P1 receptor modulator, or a pharmaceutically acceptable salt thereof, is siponimod, or a pharmaceutically acceptable salt thereof.

8) A further embodiment relates to a pharmaceutical composition according to embodiment 1), wherein the S1P1 receptor modulator, or a pharmaceutically acceptable salt thereof, is ozanimod, or a pharmaceutically acceptable salt thereof.

9) A further embodiment relates to a pharmaceutical composition according to embodiment 1), wherein the S1P1 receptor modulator, or a pharmaceutically acceptable salt thereof, is cenerimod, or a pharmaceutically acceptable salt thereof.

10) A further embodiment relates to a pharmaceutical composition according to embodiment 1), wherein the S1P1 receptor modulator, or a pharmaceutically acceptable salt thereof, is etrasimod, or a pharmaceutically acceptable salt thereof.

11) A further embodiment relates to a pharmaceutical composition according to embodiment 1), wherein the S1P1 receptor modulator, or a pharmaceutically acceptable salt thereof, is amiselimod, or a pharmaceutically acceptable salt thereof.

12) A further embodiment relates to a pharmaceutical composition according to any one of embodiments 1) to 11), wherein said S1P1 receptor modulator, or a pharmaceutically acceptable salt thereof, is comprised in a pharmaceutical dosage form suitable for the oral administration of said S1P1 receptor modulator, wherein
fingolimod, or a pharmaceutically acceptable salt thereof, if present, is comprised in said pharmaceutical dosage form in a unit dose suitable for the oral administration of a total of about 0.5 mg or below per day of fingolimod;
siponimod, or a pharmaceutically acceptable salt thereof, if present, is comprised in said pharmaceutical dosage form in a unit dose suitable for the oral administration of a total of about 2 mg or below per day of siponimod;
ponesimod, or a pharmaceutically acceptable salt thereof, if present, is comprised in said pharmaceutical dosage form in a unit dose suitable for the oral administration of a total of about 20 mg or below per day of ponesimod; and
ozanimod, or a pharmaceutically acceptable salt thereof, if present, is comprised in said pharmaceutical dosage form in a unit dose suitable for the oral administration of a total of about 1 mg or below per day of ozanimod;
cenerimod, or a pharmaceutically acceptable salt thereof, if present, is comprised in said pharmaceutical dosage form in a unit dose suitable for the oral administration of a total of about 4 mg or below per day of cenerimod;
etrasimod, or a pharmaceutically acceptable salt thereof, if present, is comprised in said pharmaceutical dosage form in a unit dose suitable for the oral administration of a total of about 2 mg or below per day of etrasimod; and
amiselimod, or a pharmaceutically acceptable salt thereof, if present, is comprised said pharmaceutical dosage form in a unit dose suitable for the oral administration of a total of about 0.4 mg or below per day of amiselimod.

The above dosage forms are especially intended for once daily (qd) dosing of said unit dose.

13) A further embodiment relates to a pharmaceutical composition according to any one of embodiments 1) to 11), wherein said S1P1 receptor modulator, or a pharmaceutically acceptable salt thereof, is comprised in a pharmaceutical dosage form suitable for the oral administration of said S1P1 receptor modulator, wherein
fingolimod, or a pharmaceutically acceptable salt thereof, if present, is comprised in said pharmaceutical dosage form in a unit dose suitable for the oral administration of a total of about 0.5 mg or below per day of fingolimod;
siponimod, or a pharmaceutically acceptable salt thereof, if present, is comprised in said pharmaceutical dosage form in a unit dose suitable for the oral administration of a total of about 2 mg or below per day of siponimod;
ponesimod, or a pharmaceutically acceptable salt thereof, if present, is comprised in said pharmaceutical dosage form in a unit dose suitable for the oral administration of a total of about 10 mg or below per day of ponesimod; and
ozanimod, or a pharmaceutically acceptable salt thereof, if present, is comprised in said pharmaceutical dosage form in a unit dose suitable for the oral administration of a total of about 0.5 mg or below per day of ozanimod;
cenerimod, or a pharmaceutically acceptable salt thereof, if present, is comprised in said pharmaceutical dosage form in a unit dose suitable for the oral administration of a total of about 2 mg or below per day of cenerimod;
etrasimod, or a pharmaceutically acceptable salt thereof, if present, is comprised in said pharmaceutical dosage form in a unit dose suitable for the oral administration of a total of about 1 mg or below per day of etrasimod; and
amiselimod, or a pharmaceutically acceptable salt thereof, if present, is comprised in said pharmaceutical dosage form in a unit dose suitable for the oral administration of a total of about 0.2 mg or below per day of amiselimod.

The above dosage forms are especially intended for once daily (qd) dosing of said unit dose.

14) A further embodiment relates to a pharmaceutical composition according to any one of embodiments 1) to 13), wherein said S1P1 receptor modulator, or a pharmaceutically acceptable salt thereof, is comprised in a dose of said S1P1 receptor modulator which is a tolerated efficacious dose or lower than a tolerated efficacious dose of said S1P1 receptor modulator when given as a single therapy (e.g. as indicated in an approval letter for such S1P1 receptor modulator for a certain disease or disorder when given as a single therapy).

15) A further embodiment relates to a pharmaceutical composition according to any one of embodiments 1) to 13), wherein said S1P1 receptor modulator, or a pharmaceutically acceptable salt thereof, is comprised in a dose of said S1P1 receptor modulator which is lower than a tolerated efficacious dose of said S1P1 receptor modulator when given as a single therapy (e.g. as indicated in an approval letter for such S1P1 receptor modulator for a certain disease or disorder when given as a single therapy).

Such combination pharmaceutical compositions according to embodiments 1) to 15) are especially useful for the prophylaxis/prevention or treatment of diseases and disorders where both CXCR7 expression or its ligands and S1P play a role and in a method for the prophylaxis/prevention or treatment of diseases and disorders where both CXCR7 expression or its ligands and S1P play a role, comprising administering a pharmaceutically effective dose of such combination pharmaceutical composition to a subject in need thereof.

Diseases and disorders where both CXCR7 expression or its ligands and S1P play a role are notably those where both CXCR7 expression or its ligands and S1P play a role (i) in the inflammatory immune response (such as migration, adhesion, survival, differentiation, polarization of cells) which takes place in a wide variety of autoimmune and inflammatory disorders, and/or (ii) in neurodegenerative processes (such as glial cell activation, proliferation, migration, neuronal survival, myelination).

In particular, diseases and disorders where CXCR7 expression or its ligands play a role are especially diseases and disorders which respond to the activation of the CXCL12 receptors and/or CXCL11 receptors; as well as diseases and disorders which respond to opioid receptor signalling.

Such diseases and disorders where CXCR7 expression or its ligands play a role are in particular defined as comprising:
cancer such as brain tumors including malignant gliomas, glioblastoma multiforme; neuroblastoma; pancreatic cancer including pancreatic adenocarcinoma/pancreatic ductal adenocarcinoma; gastro-intestinal cancers including colon carcinoma, hepatocellular carcinoma and gastric cancer; Kaposi's sarcoma; leukemias including adult T-cell leukemia; lymphoma; lung cancer; breast cancer; rhabdomyosarcoma; prostate cancer; esophageal squamous cancer; oral squamous cell carcinoma; endometrial cancer; thyroid carcinoma including papillary thyroid carcinoma; metastatic cancers; lung metastasis; skin cancer including melanoma and metastatic melanoma; bladder cancer; multiple myelomas; osteosarcoma; head and neck cancer; and renal carcinomas including renal clear cell carcinoma, and metastatic renal clear cell carcinoma;
Autoimmune and/or inflammatory diseases and disorders including especially
   autoimmune and/or inflammatory demyelinating diseases and disorders including especially
      multiple sclerosis (MS); idiopathic (inflammatory) demyelinating diseases; and autoimmune encephalomyelitis (including acute disseminated encephalomyelitis (ADEM) and multiphasic disseminated encephalomyelitis (MDEM));
      Guillain Barre syndrome; and chronic inflammatory demyelinating polyneuropathy (CIDP); and
      other autoimmune and/or inflammatory demyelinating diseases and disorders (which may be associated with the above-listed autoimmune and/or inflammatory demyelinating diseases and disorders) including especially
         neuromyelitis optica spectrum disorders (including neuromyelitis optica (Devic's disease), and (acute) optic neuritis);
         myelitis (including notably transverse myelitis spectrum disorders such as especially (acute) transverse myelitis, as well as acute flaccid myelitis, poliomyelitis, leukomyelitis, and meningococcal myelitis);
         brain stem encephalitis; and
         anti-myelin oligodendrocyte glycoprotein (anti-MOG) associated diseases (including anti-MOG encephalomyelitis);
   rheumatoid arthritis (RA);
   inflammatory bowel diseases (IBD, especially comprising Crohn's disease and ulcerative colitis);
   systemic lupus erythematosus (SLE) (including neuropsychiatric systemic lupus erythematosus and lupus nephritis);
   interstitial cystitis; celiac disease; osteoarthritis; type I diabetes; psoriasis; autoimmune thyroiditis; Sjögren's Syndrome; and vitiligo;
   chronic rhinosinusitis, asthma, chronic obstructive pulmonary disorder, atherosclerosis, myocarditis, acute lung injury, endometriosis, diabetic retinopathy and sarcoidosis;
   psoriatic arthritis; antiphospholipid syndrome; thyroiditis such as Hashimoto's thyroiditis; lymphocytic thyroiditis; myasthenia gravis; episcleritis; scleritis; Kawasaki's disease; uveo-retinitis; uveitis including posterior uveitis and uveitis associated with Behcet's disease; uveomeningitis syndrome; allergic encephalomyelitis; atopic diseases such as rhinitis, conjunctivitis, dermatitis; and post-infectious autoimmune diseases including rheumatic fever and post-infectious glomerulonephritis;
   Rasmussen's encephalitis and SUSAC syndrome (retinocochleocerebral vasculopathy);
   ankylosing spondylitis,
   juvenile idiopathic arthritis, systemic sclerosis (systemic scleroderma), giant-cell arteritis (GCA or temporal arteritis), primary biliary cholangitis (PBC or primary biliary cirrhosis); and
   cytokine release syndrome following a strong viral infection or acute respiratory distress syndrome including COVID-19;
transplant rejection including notably renal allograft rejection, cardiac allograft rejection, and graft-versus-host diseases brought about by hematopoietic stem cell transplantation;
fibrosis including notably liver fibrosis, liver cirrhosis, lung fibrosis, cardiac fibrosis; especially idiopathic pulmonary fibrosis;
ischemic injury including notably renal ischemia or cerebral ischemia;
alopecia areata, eosinophilic esophagitis, dermatomyositis/polymyositis, atopic dermatitis, and pyoderma gangrenosum;
neurodegenerative disorders including notably amyotrophic lateral sclerosis (ALS) and Huntington's disease; as well as Alzheimer's disease (AD), Parkinson's disease (PD), and adrenoleukodystrophy; and
diseases and disorders relating to opioid receptor signalling including notably neuropathic pain; as well as addiction disorders, mood disorders, and anxiety disorders.

It is understood that diseases and disorders where CXCR7 expression or its ligands play a role especially comprise autoimmune and/or inflammatory demyelinating diseases and disorders including all forms of autoimmune neuritis.

It is further understood that neuropathic pain may be associated with any other disease or disorder where CXCR7 expression or its ligands play a role.

In a further aspect of the invention, it has now been found that COMPOUND or a pharmaceutically acceptable salt thereof, when administered as a single active ingredient, may be used for the prophylaxis/prevention and treatment of said “diseases and disorders where CXCR7 expression or its ligands play a role” as defined herein above, wherein COMPOUND preferably is used/administered/to be administered in a particular pharmacologically effective dosing regimen. COMPOUND may be used alone (i.e. as a single active ingredient), especially in such preferred particular dosing, for the prophylaxis/prevention and treatment of said diseases and disorders; or COMPOUND may be used, especially in such preferred particular dosing regimen, in combination with an S1P1 receptor modulator [e.g. in a fixed dose combination according to in any one of embodiments 1) to 15); or in an equivalent non-fixed dose combination], wherein when used in combination, said “diseases and disorders where CXCR7 expression or its ligands play a role” are such that both CXCR7 expression or its ligands and S1P play a role (such diseases and disorders as defined herein).

Such particular dosage regimen may comprise administering COMPOUND, or a pharmaceutically acceptable salt thereof, in a total dose of between about 20 mg to about 300 mg per day, wherein said total dose is given/administered especially in one unit dose (once per day=quaque die=qd), or in two separate unit doses (twice daily=bis in die=bid). For example said total dose may be achieved by dosing between about 20 mg qd to about 300 mg qd, or between about 10 mg bid to about 150 mg bid.

Notably, such dosage regimen may comprise administering COMPOUND in a total dose of about 20 mg to 300 mg per day, about 20 mg to 200 mg per day, about 30 mg to 150 mg per day, about 40 mg to 150 mg per day, about 50 mg to 200 mg, about 50 mg to 100 mg, about 100 mg to 200 mg, or especially about 75 mg to 150 mg per day; wherein said total dose is given/administered especially in one unit dose (qd), or in two separate unit doses (bid). Examples of such dosage regimen comprise administering of a total dose of about 200 mg, about 150 mg, about 100 mg, about 75 mg, about 50 mg, about 30 mg, or about 25 mg per day given in one unit dose (qd) of COMPOUND, or in two separate unit doses (bid) of COMPOUND, wherein particular examples of such twice daily dosing would comprise for example administering of about 100 mg bid, about 75 mg bid, about 50 mg bid, about 25 mg bid, or about 15 mg bid of COMPOUND.

For avoidance of doubt, for the present invention any amount/unit dose in mg of COMPOUND refers to the amount/unit dose suitable for the administration of COMPOUND in free base form having a molecular weight of 522.56 g/mol in such amount/unit dose. Such amount/unit dose may need to be adjusted in a pharmaceutical composition in case COMPOUND is present in such composition in a form different from anhydrous free base, such as a in form of a pharmaceutically acceptable salt; and/or a solvate such as a hydrate. In case the active ingredient is administered e.g. in form of a pharmaceutically acceptable salt, it is understood that the respective amount of active pharmaceutical ingredient (e.g. said pharmaceutically acceptable salt) in a pharmaceutical composition will be adapted accordingly.

A certain dosage form/dosage regimen generally is considered equivalent (bioequivalent as per FDA guidelines) in case it reaches a maximal concentration Cₘₐₓ of active ingredient between 80% to 125%, and an exposure of active ingredient expressed as area under the curve (AUC) between 80% to 125%, of the respective values in plasma achieved with a given dosage form and dosage regimen.
i) One particular aspect of the present invention, thus, relates to COMPOUND, or a pharmaceutically acceptable salt thereof, for use in the prevention/prophylaxis or treatment of “diseases and disorders where CXCR7 expression or its ligands play a role” (as defined herein), wherein COMPOUND is (to be) administered in a total dose of between about 20 mg to about 300 mg (notably about 20 mg to about 200 mg; especially about 50 mg to about 150 mg) per day of COMPOUND. In a sub-embodiment, said total dose is given/administered especially in one unit dose per day [qd; for example between about 20 mg qd to about 300 mg qd (notably about 20 mg qd to about 200 mg qd; especially about 50 mg qd to about 150 mg qd)], or in two separate unit doses per day [bid; for example between about 10 mg bid to about 150 mg bid (notably about 10 mg bid to about 100 mg bid; especially about 25 mg bid to about 75 mg bid)].
   i)(a) In a sub-embodiment, such diseases and disorders where CXCR7 expression or its ligands play a role especially comprise:
      an autoimmune and/or inflammatory disease and disorder as defined herein; wherein said disease or disorder especially is
         an autoimmune and/or inflammatory demyelinating disease or disorder, including in particular multiple sclerosis (MS), idiopathic inflammatory demyelinating diseases, neuromyelitis optica spectrum diseases (including neuromyelitis optica and (acute) optic neuritis), auto-immune encephalomyelitis (including acute disseminated encephalomyelitis (ADEM) and multiphasic disseminated encephalomyelitis (MDEM)), myelitis (including notably transverse myelitis spectrum disorders such as especially (acute) transverse myelitis, as well as acute flaccid myelitis, poliomyelitis, leukomyelitis, and meningococcal myelitis), brain stem encephalitis, anti-myelin oligodendrocyte glycoprotein (anti-MOG) associated diseases (including anti-MOG encephalomyelitis); Guillain-Barre syndrome, chronic inflammatory demyelinating polyneuropathy (CIDP), and anti-myelin-associated glycoprotein (anti-MAG) peripheral neuropathy;
         rheumatoid arthritis (RA);
         an inflammatory bowel disease (IBD); in particular Crohn's disease or ulcerative colitis;
         systemic lupus erythematosus (SLE) including neuropsychiatric systemic lupus erythematosus and lupus nephritis;
         interstitial cystitis;
         celiac disease;
         osteoarthritis;
         psoriasis;
         type I diabetes;
         ankylosing spondylitis; or
         cytokine release syndrome following a strong viral infection or acute respiratory distress syndrome including COVID-19;
      transplant rejection including notably renal allograft rejection, cardiac allograft rejection, and graft-versus-host diseases brought about by hematopoietic stem cell transplantation; or
      a neurodegenerative disorder including notably amyotrophic lateral sclerosis (ALS) and Huntington's disease; as well as Alzheimer's disease (AD), Parkinson's disease (PD), and adrenoleukodystrophy.
   i)(b) In another sub-embodiment, such diseases and disorders where CXCR7 expression or its ligands play a role especially comprise fibrosis including notably liver fibrosis, liver cirrhosis, lung fibrosis, cardiac fibrosis; especially idiopathic pulmonary fibrosis.
   i)(c) In another sub-embodiment, such diseases and disorders where CXCR7 expression or its ligands play a role especially comprise ischemic injury including notably renal ischemia or cerebral ischemia.
   i)(d) In another sub-embodiment, such diseases and disorders where CXCR7 expression or its ligands play a role especially comprise diseases or disorders relating to opioid receptor signalling including notably neuropathic pain; as well as addiction disorders, mood disorders, and anxiety disorders.
   i)(e) In another sub-embodiment, such diseases and disorders where CXCR7 expression or its ligands play a role especially comprise cancer such as brain tumors including malignant gliomas, glioblastoma multiforme; neuroblastoma; pancreatic cancer including pancreatic adenocarcinoma/pancreatic ductal adenocarcinoma; gastro-intestinal cancers including colon carcinoma, hepatocellular carcinoma and gastric cancer; Kaposi's sarcoma; leukemias including adult T-cell leukemia; lymphoma; lung cancer; breast cancer; rhabdomyosarcoma; prostate cancer; esophageal squamous cancer; oral squamous cell carcinoma; endometrial cancer; thyroid carcinoma including papillary thyroid carcinoma; metastatic cancers; lung metastasis; skin cancer including melanoma and metastatic melanoma; bladder cancer; multiple myelomas; osteosarcoma; head and neck cancer; and renal carcinomas including renal clear cell carcinoma, metastatic renal clear cell carcinoma.
ii) A second particular aspect of the present invention, relates to COMPOUND, or a pharmaceutically acceptable salt thereof, for use according to embodiment i) (or any one of its sub-embodiments), wherein COMPOUND is (to be) administered in a total dose of about 20 to 200 mg per day of COMPOUND; notably about 30 mg to 150 mg per day, about 40 mg to 150 mg per day, about 50 mg to 200 mg, about 50 mg to 150 mg, about 50 mg to 100 mg, or about 100 mg to 200 mg per day of COMPOUND; especially about 75 mg to 150 mg per day of COMPOUND. In a sub-embodiment, said total dose is given/administered especially in one unit dose per day (qd), or in two separate unit doses per day (bid).
iii) A third particular aspect of the present invention, relates to COMPOUND, or a pharmaceutically acceptable salt thereof, for use according to embodiment i) (or any one of its sub-embodiments), wherein COMPOUND is (to be) administered in a total dose of about 200 mg, about 150 mg, about 100 mg, about 75 mg, about 50 mg, or about 30 mg per day of COMPOUND; especially about 150 mg, about 100 mg, or about 75 mg per day of COMPOUND. In a sub-embodiment, said total dose is given/administered especially in one unit dose per day (qd), or in two separate unit doses per day (bid).
iv) A fourth particular aspect of the present invention, relates to COMPOUND, or a pharmaceutically acceptable salt thereof, for use according to embodiment i) (or any one of its sub-embodiments), wherein COMPOUND is (to be) administered in a total dose of about 200 mg, about 150 mg, about 125 mg, about 100 mg, about 75 mg, about 50 mg, about 30 mg, or about 25 mg per day of COMPOUND; especially about 150 mg, about 125 mg, about 100 mg, or about 75 mg per day of COMPOUND; wherein said total dose is given/administered in one unit dose per day (qd).
v) A fifth particular aspect of the present invention, relates to COMPOUND, or a pharmaceutically acceptable salt thereof, for use according to embodiment i) (or any one of its sub-embodiments), wherein COMPOUND is (to be) administered in a total dose of about 200 mg given/administered about 100 mg bid, about 150 mg given/administered about 75 mg bid, about 120 mg given/administered about 60 mg bid, about 100 mg given/administered about 50 mg bid, about 80 mg given/administered about 40 mg bid, about 60 mg given/administered about 30 mg bid, about 50 mg given/administered about 25 mg bid, or about 30 mg given/administered about 15 mg bid per day of COMPOUND; especially about 150 mg given/administered about 75 mg bid, or about 100 mg given/administered about 50 mg bid per day of COMPOUND.

COMPOUND can be used as medicament as a single active ingredient (optionally in combination with an S1P1 receptor modulator, i.e. without being combined with such S1P1 receptor modulator, or in combination with such S1P1 receptor modulator) according to the present invention, e.g. in the form of pharmaceutical compositions especially for enteral, or for parenteral administration.
vi) Another aspect of the invention, thus, relates to a pharmaceutical composition comprising COMPOUND, or a pharmaceutically acceptable salt thereof, wherein COMPOUND is comprised in a unit dose suitable for administration of COMPOUND in a total dose per day as defined in any one of embodiments i) to v).
viii) A further aspect of the invention relates to COMPOUND, or a pharmaceutically acceptable salt thereof, for use according to any one of embodiments i) to v), wherein COMPOUND is used for the prophylaxis/prevention or treatment of a disease and disorder where both CXCR7 expression or its ligands and S1P play a role; wherein the characteristics of embodiments 16) to 35) herein below apply mutatis mutandis.
vii) Another aspect of the invention relates to a pharmaceutical composition comprising, as active principles, COMPOUND, or a pharmaceutically acceptable salt thereof, in combination with an S1P1 receptor modulator, or a pharmaceutically acceptable salt thereof, according to any one of embodiments 1) to 15), wherein COMPOUND is comprised in a unit dose suitable for administration of COMPOUND in a total dose per day as defined in any one of embodiments i) to v); wherein such composition is notably for once daily (qd) dosing/administration.
viii) A further aspect of the invention relates to COMPOUND, or a pharmaceutically acceptable salt thereof, for use according to any one of embodiments i) to v), wherein COMPOUND is for use in the prophylaxis/prevention or treatment of a disease and disorder where both CXCR7 expression or its ligands and S1P play a role; wherein COMPOUND is intended to be used/administered/(intended) to be administered in combination with an S1P1 receptor modulator, or a pharmaceutically acceptable salt thereof; wherein the characteristics of any one of embodiments 16) to 48) herein below apply mutatis mutandis.

Diseases and disorders where both CXCR7 expression or its ligands and S1P play a role are such diseases and disorders where CXCR7 expression or its ligands play a role which preferably have a component of an inflammatory immune response.

Such diseases and disorders where both CXCR7 expression or its ligands and S1P play a role may in particular be defined as including autoimmune and/or inflammatory diseases and disorders, transplant rejection, and neurodegenerative diseases and disorders; especially autoimmune diseases and disorders which have an inflammatory component, in particular autoimmune and/or inflammatory demyelinating diseases and disorders.

The term “transplant rejection” may be defined as comprising rejection of transplanted organs such as kidney, liver, heart, lung, pancreas, cornea, and skin; graft-versus-host diseases brought about by hematopoietic stem cell transplantation; chronic allograft rejection and chronic allograft vasculopathy.

The term “neurodegenerative diseases and disorders” may be defined as comprising especially neurodegenerative diseases and disorders where both CXCR7 expression or its ligands and S1P play a role in the neurodegeneration (e.g. glial cell activation, neuronal survival, myelination) associated with such disease and disorder. Particular examples include amyotrophic lateral sclerosis (ALS), Alzheimer's disease (AD), Parkinson's disease (PD), Huntington's disease, and adrenoleukodystrophy.

The term “autoimmune and/or inflammatory diseases and disorders” refers in particular to any autoimmune and/or inflammatory disease or disorder where both CXCR7 expression or its ligands and S1P play a role, especially to autoimmune diseases and disorders which have an inflammatory component. Examples of such autoimmune and/or inflammatory diseases and disorders comprise autoimmune and/or inflammatory demyelinating diseases and disorders including all forms of autoimmune neuritis. In particular, autoimmune and/or inflammatory demyelinating diseases and disorders include multiple sclerosis (MS), Guillain Barre syndrome, chronic inflammatory demyelinating polyneuropathy (CIDP), and other autoimmune and/or inflammatory demyelinating diseases and disorders (which may be associated with the above-listed autoimmune and/or inflammatory demyelinating diseases and disorders) such as neuromyelitis optica spectrum disorders (including neuromyelitis optica (Devic's disease), and (acute) optic neuritis), auto-immune encephalomyelitis (including acute disseminated encephalomyelitis (ADEM) and multiphasic disseminated encephalomyelitis (MDEM)), myelitis (including notably transverse myelitis spectrum disorders such as especially (acute) transverse myelitis, as well as acute flaccid myelitis, poliomyelitis, leukomyelitis, and meningococcal myelitis), brain stem encephalitis, and anti-myelin oligodendrocyte glycoprotein (anti-MOG) associated diseases (including anti-MOG encephalomyelitis); rheumatoid arthritis (RA); inflammatory bowel disease (IBD, especially comprising Crohn's disease and ulcerative colitis); systemic lupus erythematosus (SLE) (including neuropsychiatric systemic lupus erythematosus and lupus nephritis); interstitial cystitis; celiac disease; osteoarthritis; psoriasis; and type I diabetes. In addition, autoimmune and inflammatory diseases and disorders further comprise disorders such as psoriatic arthritis; antiphospholipid syndrome; thyroiditis such as Hashimoto's thyroiditis; lymphocytic thyroiditis; myasthenia gravis; episcleritis; scleritis; Kawasaki's disease; uveo-retinitis; uveitis including posterior uveitis, and uveitis associated with Behcet's disease; uveomeningitis syndrome; allergic encephalomyelitis; atopic diseases such as rhinitis, conjunctivitis, dermatitis; and post-infectious autoimmune diseases including rheumatic fever and post-infectious glomerulonephritis.

In addition to the above-listed, further autoimmune and/or inflammatory diseases or disorders where both CXCR7 expression or its ligands and S1P play a role include autoimmune and/or inflammatory demyelinating diseases and disorders such as Rasmussen's encephalitis and SUSAC syndrome (retinocochleocerebral vasculopathy); as well as other autoimmune and inflammatory diseases and disorders such as ankylosing spondylitis, juvenile idiopathic arthritis, systemic sclerosis (systemic scleroderma), giant-cell arteritis (GCA or temporal arteritis), primary biliary cholangitis (PBC or primary biliary cirrhosis); and cytokine release syndrome following a strong viral infection or acute respiratory distress syndrome including COVID-19.

Particular examples of autoimmune and/or inflammatory diseases and disorders are
autoimmune and/or inflammatory demyelinating diseases and disorders including notably multiple sclerosis (MS), Guillain Barre syndrome, chronic inflammatory demyelinating polyneuropathy (CIDP), and other autoimmune and/or inflammatory demyelinating diseases and disorders;
rheumatoid arthritis (RA);
inflammatory bowel disease (IBD), including especially Crohn's disease and ulcerative colitis;
systemic lupus erythematosus (SLE) (including neuropsychiatric systemic lupus erythematosus and lupus nephritis); and, in addition to the above-listed,
ankylosing spondylitis, and
cytokine release syndrome following a strong viral infection or acute respiratory distress syndrome including COVID-19.
In a first sub-embodiment, the term “autoimmune and/or inflammatory diseases and disorders” especially refers to autoimmune and/or inflammatory demyelinating diseases and disorders including especially multiple sclerosis (MS), Guillain Barre syndrome, chronic inflammatory demyelinating polyneuropathy (CIDP), and other autoimmune and/or inflammatory demyelinating diseases and disorders as defined herein such as notably auto-immune encephalomyelitis and myelitis.
In a second sub-embodiment, the term “autoimmune and/or inflammatory diseases and disorders” refers to inflammatory bowel diseases including especially Crohn's disease and ulcerative colitis.
In a third sub-embodiment, the term “autoimmune and/or inflammatory diseases and disorders” refers to systemic lupus erythematosus (SLE) including neuropsychiatric systemic lupus erythematosus and lupus nephritis.
In a fourth sub-embodiment, the term “autoimmune and/or inflammatory diseases and disorders” refers to ankylosing spondylitis.
In a fifth sub-embodiment, the term “autoimmune and/or inflammatory diseases and disorders” refers to cytokine release syndrome following a strong viral infection or acute respiratory distress syndrome including COVID-19.

The term “autoimmune and/or inflammatory demyelinating diseases and disorders” refers to demyelinating diseases and disorders of the central nervous system such as especially multiple sclerosis (MS), as well as idiopathic inflammatory demyelinating diseases, neuromyelitis optica spectrum diseases (including neuromyelitis optica (Devic's disease) and (acute) optic neuritis), auto-immune encephalomyelitis (including acute disseminated encephalomyelitis (ADEM) and multiphasic disseminated encephalomyelitis (MDEM)), myelitis (including notably transverse myelitis spectrum disorders such as especially (acute) transverse myelitis, as well as acute flaccid myelitis, poliomyelitis, leukomyelitis, and meningococcal myelitis), brain stem encephalitis, and anti-myelin oligodendrocyte glycoprotein (anti-MOG) associated diseases (including anti-MOG encephalomyelitis); as well as autoimmune and/or inflammatory demyelinating diseases and disorders of the peripheral nervous system including especially Guillain-Barre syndrome and its chronic counterpart chronic inflammatory demyelinating polyneuropathy (CIDP, alternatively named chronic relapsing polyneuropathy (CRP)), and anti-myelin-associated glycoprotein (anti-MAG) peripheral neuropathy.
In a first sub-embodiment, the term “autoimmune and/or inflammatory demyelinating diseases and disorders” refers especially to multiple sclerosis (MS), Guillain Barre syndrome, chronic inflammatory demyelinating polyneuropathy (CIDP), and other autoimmune and/or inflammatory demyelinating diseases and disorders as defined herein such as notably auto-immune encephalomyelitis and myelitis.
In a second sub-embodiment, the term “autoimmune and/or inflammatory demyelinating diseases and disorders” notably refers to myelitis which is a transverse myelitis spectrum disorder such as especially (acute) transverse myelitis;
wherein said transverse myelitis spectrum disorder may be
   idiopathic (without known cause), or
   caused by/associated with multiple sclerosis, or
   caused by/associated with SLE, neuromyelitis optica spectrum disorders, antiphospholipid syndrome, or other autoimmune and inflammatory diseases and disorders as defined herein;
   or caused by/associated with infectious diseases such as infection by viruses, bacteria, mold, or parasites
   [especially including bacterial infection (e.g. with Mycoplasma pneumoniae, Bartonella henselae, borrelia (Lyme disease), Campylobacter jejuni, syphilis, tuberculosis (TB)); and viral infection (e.g. viral meningoencephalitis (meningitis), or infection with HIV, herpes simplex, herpes zoster, cytomegalovirus, Epstein-Barr virus, flaviviridae such as Zika virus and West Nile virus)];
   or caused by/associated with vaccination (including vaccination against coronaviruses such as SARS-CoV/SARS-CoV-1).
In a third sub-embodiment, the term “autoimmune and/or inflammatory demyelinating diseases and disorders” notably refers to neuromyelitis optica spectrum disorders such as especially (acute) optic neuritis, wherein said neuromyelitis optica spectrum disorders may be
   idiopathic (without known cause),
   or caused by/associated with multiple sclerosis, or
   caused by/associated with SLE, or other autoimmune and/or inflammatory diseases and disorders; or
   caused by/associated with infectious diseases including especially Lyme disease.
Likewise, in a fourth sub-embodiment, the term “autoimmune and/or inflammatory demyelinating diseases and disorders” notably refers to any autoimmune and/or inflammatory demyelinating disease or disorder, such as especially MS, which is associated with neuromyelitis optica spectrum disorders such as especially (acute) optic neuritis.

The term “idiopathic inflammatory demyelinating disease” refers to an inflammatory demyelinating disease of unknown etiology; especially to variants or borderline forms of multiple sclerosis differing for example in terms of chronicity, severity, and clinical course.

A particular example of an “autoimmune and/or inflammatory diseases and disorders” is the autoimmune demyelinating disease multiple sclerosis (MS), wherein it is understood that MS can be further classified as relapsing remitting MS, primary progressive MS or secondary progressive MS.

A particular feature of autoimmune and/or inflammatory demyelinating diseases and disorders such as especially MS relates to the demyelination aspect present such disease or disorder. Therefore, one aspect of the present invention relates to the treatment of an autoimmune and/or inflammatory demyelinating disease or disorder such as especially MS, wherein the rate of progression of said disease or disorder is reduced, especially the rate of progression of demyelination and/or the rate of appearance of irreversible neurodegenerative damage such as axonal damage is reduced. An additional aspect of the present invention relates to a treatment of an autoimmune and/or inflammatory demyelinating disease or disorder such as especially MS, wherein the said treatment has an effect in/results in a remyelination.

The term “Clinically Isolated Syndrome” (CIS) refers to a first episode of neurologic symptoms that lasts at least 24 h and is caused by inflammation or demyelination in the central nervous system (CNS). The episode generally is characteristic of an autoimmune and/or inflammatory demyelinating disease or disorder, in particular MS, however, patients diagnosed as having experienced a CIS may or may not subsequently develop an autoimmune and/or inflammatory demyelinating disease or disorder, in particular MS. When a CIS is accompanied by lesions identified e.g. by means of a brain MRI (magnetic resonance imaging) that are similar to those seen in MS, the person has a high likelihood of a second episode of neurologic symptoms and diagnosis of relapsing-remitting MS. When CIS is not accompanied by MS-like lesions on a brain MRI, the person has a much lower likelihood of developing MS. The diagnostic criteria for MS (see for example the 2018 Revised Guidelines: https://www.mscare.org/page/MRI_protocol) make it possible to diagnose MS in a patient having experienced a CIS who also has specific findings on brain MRI that provide evidence of an earlier episode of damage in a different location and indicate active inflammation in a region other than the one causing the current symptoms. Individuals with CIS may be considered as being at high risk for developing MS. In the U.S. such patients may be treated with a disease-modifying therapy that has been approved by the U.S. Food and Drug Administration (FDA) for that purpose. Early treatment of CIS has been shown to delay onset of an autoimmune and/or inflammatory demyelinating disease or disorder, in particular MS.

The term prevention/prophylaxis of an autoimmune and/or inflammatory demyelinating disease or disorder thus especially includes delaying the onset of such autoimmune and/or inflammatory demyelinating disease or disorder [e.g. by preventing demyelination and/or by remyelination of initially occurring demyelination (such as in a Clinically Isolated Syndrome)].

Therefore, another aspect of the present invention relates to the pharmaceutical compositions according to embodiments 1) to 15) for the prevention/prophylaxis of an autoimmune and/or inflammatory demyelinating disease or disorder, in particular MS, wherein said prevention/prophylaxis of an autoimmune and/or inflammatory demyelinating disease or disorder, in particular MS comprises delaying the onset of said autoimmune and/or inflammatory demyelinating disease or disorder, in particular MS in a patient who has experienced a CIS/has been diagnosed as having experienced a CIS.

In a particular embodiment of the present invention, said combination pharmaceutical compositions according to embodiments 1) to 15) prevent or treat demyelination; wherein especially the subject to be treated has been diagnosed as having an autoimmune and/or inflammatory demyelinating diseases and disorders as defined herein.

In a further particular embodiment of the present invention, said combination pharmaceutical compositions according to embodiments 1) to 15) prevent or treat demyelination in a patient, wherein said prevention or treatment of demyelination additionally includes a remyelinating effect; wherein especially the subject to be treated has been diagnosed as having an autoimmune and/or inflammatory demyelinating diseases and disorders as defined herein.

16) A second aspect of the invention relates to COMPOUND, or a pharmaceutically acceptable salt thereof, for use in the prophylaxis/prevention or treatment of a disease and disorder where both CXCR7 expression or its ligands and S1P play a role, especially in the prophylaxis/prevention or treatment of autoimmune and inflammatory diseases and disorders, transplant rejection, and neurodegenerative diseases and disorders (notably autoimmune and/or inflammatory diseases and disorders; especially autoimmune diseases and disorders which have an inflammatory component, in particular autoimmune and/or inflammatory demyelinating diseases and disorders); wherein COMPOUND is (intended) (to be) administered in combination with an S1P1 receptor modulator, or a pharmaceutically acceptable salt thereof.

17) A further embodiment relates to COMPOUND, or a pharmaceutically acceptable salt thereof, for use according to embodiment 16); wherein such use is for the treatment of
an autoimmune and/or inflammatory disease and disorder; wherein said disease or disorder especially is
   an autoimmune and/or inflammatory demyelinating disease or disorder, including in particular multiple sclerosis (MS); idiopathic inflammatory demyelinating diseases; neuromyelitis optica spectrum diseases including neuromyelitis optica and (acute) optic neuritis; auto-immune encephalomyelitis including acute disseminated encephalomyelitis (ADEM) and multiphasic disseminated encephalomyelitis (MDEM); myelitis including notably transverse myelitis spectrum disorders such as especially (acute) transverse myelitis, as well as acute flaccid myelitis, poliomyelitis, leukomyelitis, and meningococcal myelitis; brain stem encephalitis; anti-myelin oligodendrocyte glycoprotein (anti-MOG) associated diseases including anti-MOG encephalomyelitis; Guillain-Barre syndrome; chronic inflammatory demyelinating polyneuropathy (CIDP); and anti-myelin-associated glycoprotein (anti-MAG) peripheral neuropathy;
   rheumatoid arthritis (RA);
   an inflammatory bowel disease (IBD); in particular Crohn's disease or ulcerative colitis;
   systemic lupus erythematosus (SLE) including neuropsychiatric systemic lupus erythematosus and lupus nephritis;
   interstitial cystitis;
   celiac disease;
   osteoarthritis;
   psoriasis;
   type I diabetes; or, in addition to the above-listed,
   ankylosing spondylitis; or
   cytokine release syndrome following a strong viral infection or acute respiratory distress syndrome including COVID-19;
a transplant rejection; wherein said transplant rejection especially is rejection of a transplanted organ such as kidney, liver, heart, lung, pancreas, cornea, or skin; graft-versus-host disease brought about by hematopoietic stem cell transplantation; chronic allograft rejection and chronic allograft vasculopathy; or
a neurodegenerative disease and disorder; wherein said neurodegenerative disease and disorder especially is amyotrophic lateral sclerosis (ALS), or Huntington's disease; or in addition, Alzheimer's disease (AD), Parkinson's disease (PD), or adrenoleukodystrophy.

18) A further embodiment relates to COMPOUND, or a pharmaceutically acceptable salt thereof, for use according to embodiment 16), wherein said disease and disorder where both CXCR7 expression or its ligands and S1P play a role is an autoimmune and/or inflammatory disease or disorder selected from:
an autoimmune and/or inflammatory demyelinating disease or disorder especially selected from multiple sclerosis (MS); idiopathic inflammatory demyelinating diseases; auto-immune encephalomyelitis including acute disseminated encephalomyelitis (ADEM) and multiphasic disseminated encephalomyelitis (MDEM); Guillain-Barré syndrome; chronic inflammatory demyelinating polyneuropathy (CIDP); anti-myelin-associated glycoprotein (anti-MAG) peripheral neuropathy; and myelin oligodendrocyte glycoprotein (MOG)-antibody associated disease; as well as myelitis including notably transverse myelitis spectrum disorders such as especially (acute) transverse myelitis, as well as acute flaccid myelitis, poliomyelitis, leukomyelitis, and meningococcal myelitis;
an inflammatory bowel disease especially selected from Crohn's disease and ulcerative colitis; or
systemic lupus erythematosus (SLE) including neuropsychiatric systemic lupus erythematosus and lupus nephritis.

The term “prophylaxis/prevention or treatment” in the context of the diseases and disorders defined herein, notably according to embodiments 16), 17) and 18), especially refers to the treatment of said diseases and disorders; wherein for chronic progressive diseases and disorders (including primary or secondary progressive and relapsing-remitting) the term “treatment” in particular refers to a reduction of the rate of progression of said diseases or disorders. Such reduction of the rate of progression can for example be expressed by a reduced rate of disability progression; a reduced rate of irreversible neurodegenerative damage such as axonal damage; a reduced rate of demyelination; or, in case said disease or disorder is related to the brain/the central nervous system, a reduced rate of brain atrophy/cerebral atrophy (such as especially diagnosed by magnetic resonance imaging (MRI)).

In another aspect the term “prophylaxis/prevention or treatment” in the context of the diseases and disorders defined herein, notably according to embodiments 16), 17) and 18), especially also refers to the prophylaxis/prevention of said diseases and disorders, in particular to delaying the onset of said diseases or disorders in subjects who are at risk/have been diagnosed as being at risk of developing such disease or disorder; e.g. a subject who has experienced a clinically isolated syndrome (CIS)/has been diagnosed as having experienced a CIS, such CIS being generally know as indicating that such subject may be at risk. Such delay of the onset can for example be expressed by an increase of time until a diagnosis of said disease or disorder can be established; in particular it can be expressed by increase of time until disability; by increase of time until first relapse, if applicable; an increase of time until diagnosis of (progressing) irreversible neurodegenerative damage such as axonal damage; an increase of time until diagnosis of demyelination; or, in case said disease or disorder is related to the brain/the central nervous system, an increase of time until diagnosis of progressing brain atrophy/cerebral atrophy (such as especially diagnosed by magnetic resonance imaging (MRI)).

19) A further embodiment relates to COMPOUND, or a pharmaceutically acceptable salt thereof, for use in the prophylaxis/prevention or treatment of a neurodegenerative disease or disorder; wherein said neurodegenerative disease or disorder especially is amyotrophic lateral sclerosis (ALS) or Huntington's disease; or Alzheimer's disease (AD), Parkinson's disease (PD), or adrenoleukodystrophy; wherein COMPOUND is (intended) to be administered in combination with an S1P1 receptor modulator, or a pharmaceutically acceptable salt thereof.

20) A further embodiment relates to COMPOUND, or a pharmaceutically acceptable salt thereof, for use according to embodiment 19) wherein such use is for the treatment of a patient diagnosed with said neurodegenerative disease or disorder, wherein said treatment reduces the rate of progression of said neurodegenerative disease or disorder.

21) A further embodiment relates to COMPOUND, or a pharmaceutically acceptable salt thereof, for use according to embodiment 20); wherein such reduced rate of progression of said neurodegenerative disease or disorder may be expressed by a reduced rate of brain atrophy/cerebral atrophy (such as especially diagnosed by magnetic resonance imaging (MRI)).

22) A further embodiment relates to COMPOUND, or a pharmaceutically acceptable salt thereof, for use according to any one of embodiments 16) to 18), wherein COMPOUND is for use in the prophylaxis/prevention or treatment of an autoimmune and/or inflammatory demyelinating disease or disorder;
wherein notably such autoimmune and/or inflammatory demyelinating disease or disorder is
   multiple sclerosis (MS);
   idiopathic inflammatory demyelinating diseases;
   neuromyelitis optica spectrum diseases (including neuromyelitis optica and (acute) optic neuritis);
   auto-immune encephalomyelitis (including acute disseminated encephalomyelitis (ADEM) and multiphasic disseminated encephalomyelitis (MDEM));
   myelitis (including notably transverse myelitis spectrum disorders such as especially (acute) transverse myelitis, as well as acute flaccid myelitis, poliomyelitis, leukomyelitis, and meningococcal myelitis);
   brain stem encephalitis;
   anti-myelin oligodendrocyte glycoprotein (anti-MOG) associated diseases (including anti-MOG encephalomyelitis);
   Guillain-Barre syndrome;
   chronic inflammatory demyelinating polyneuropathy (CIDP); or
   anti-myelin-associated glycoprotein (anti-MAG) peripheral neuropathy.

23) A further embodiment relates to COMPOUND, or a pharmaceutically acceptable salt thereof, for use according to embodiment 22), wherein such use is for the treatment of a patient who has been diagnosed with an autoimmune and/or inflammatory demyelinating disease or disorder, wherein said treatment reduces the rate of progression of said autoimmune and/or inflammatory demyelinating disease or disorder; wherein notably such autoimmune and/or inflammatory demyelinating disease or disorder is as listed in embodiment 22).

24) A further embodiment relates to COMPOUND, or a pharmaceutically acceptable salt thereof, for use according to embodiment 22), wherein such use is for the prophylaxis/prevention of said autoimmune and/or inflammatory demyelinating disease or disorder; wherein the onset of said autoimmune and/or inflammatory demyelinating disease or disorder is delayed in a subject who is at risk/has been diagnosed as being at risk of developing such disease or disorder; wherein notably such autoimmune and/or inflammatory demyelinating disease or disorder is as listed in embodiment 22).

25) A further embodiment relates to COMPOUND, or a pharmaceutically acceptable salt thereof, for use according to embodiment 23); wherein such reduced rate of progression of said autoimmune and/or inflammatory demyelinating disease or disorder may be expressed by a reduced rate of demyelination and/or a reduced rate of irreversible neurodegenerative damage such as axonal damage.

26) A further embodiment relates to COMPOUND, or a pharmaceutically acceptable salt thereof, for use according to embodiment 23) or 25); wherein such reduced rate of progression of said autoimmune and/or inflammatory demyelinating disease or disorder may especially be expressed by a reduced rate of disability progression.

27) A further embodiment relates to COMPOUND, or a pharmaceutically acceptable salt thereof, for use according to embodiment 23), 25) or 26); wherein such reduced rate of progression of said autoimmune and/or inflammatory demyelinating disease or disorder may be expressed by a reduced rate of brain atrophy/cerebral atrophy (such as especially diagnosed by magnetic resonance imaging (MRI)); wherein it is understood that said autoimmune and/or inflammatory demyelinating disease or disorder is in particular MS, notably relapsing-remitting multiple sclerosis (RRMS), secondary progressive multiple sclerosis (SPMS), or primary progressive multiple sclerosis (PPMS).

28) A further embodiment relates to COMPOUND, or a pharmaceutically acceptable salt thereof, for use according to embodiment 24), wherein said delay of the onset of said autoimmune and/or inflammatory demyelinating disease or disorder is expressed
by an increase of time until a diagnosis of said disease or disorder can be established;
by increase of time until disability;
in case said disease or disorder is characterized by a disease progression comprising relapses, by an increase of time until first relapse;
by an increase of time until diagnosis of (progressing) (irreversible) neurodegenerative damage such as axonal damage;
by an increase of time until diagnosis of demyelination; or
in case said disease or disorder is related to the brain/the central nervous system, by an increase of time until diagnosis of progressing brain atrophy/cerebral atrophy (such as especially diagnosed by magnetic resonance imaging (MRI)).

29) A further embodiment relates to COMPOUND, or a pharmaceutically acceptable salt thereof, for use according to any one of embodiments 22) to 28), wherein said prophylaxis/prevention or treatment induces an effect of remyelination.

An effect of remyelination may be expressed for example as tissue repair (e.g. of the extracellular matrix). Such effect of remyelination may for example be visualized by well known magnetic resonance imaging (MRI) techniques including magnetization transfer imaging (MTI), and notably diffusion-weighted magnetic resonance imaging (DWI or DW-MRI), especially diffusion tensor imaging (DTI).

30) A further embodiment relates to COMPOUND, or a pharmaceutically acceptable salt thereof, for use according to embodiment 22), wherein such use is for the treatment of a patient diagnosed with MS; wherein in particular said treatment reduces the rate of progression of MS, wherein such reduced rate of progression of MS may especially be expressed by a reduced rate of demyelination and/or a reduced rate of irreversible neurodegenerative damage such as axonal damage; wherein it is understood that such MS may notably be relapsing-remitting multiple sclerosis (RRMS), secondary progressive multiple sclerosis (SPMS), or primary progressive multiple sclerosis (PPMS).

31) A further embodiment relates to COMPOUND, or a pharmaceutically acceptable salt thereof, for use according to embodiment 22), wherein such use is for the treatment of a patient diagnosed with MS; wherein in particular said treatment reduces the rate of progression of MS, wherein such reduced rate of progression of MS may especially be expressed by a reduced rate of disability progression; wherein it is understood that such MS may notably be relapsing-remitting multiple sclerosis (RRMS), secondary progressive multiple sclerosis (SPMS), or primary progressive multiple sclerosis (PPMS).

32) A further embodiment relates to COMPOUND, or a pharmaceutically acceptable salt thereof, for use according to embodiment 22), wherein such use is for the treatment of a patient diagnosed with MS; wherein in particular said treatment reduces the rate of progression of MS, wherein such reduced rate of progression of MS may especially be expressed by a reduced rate of brain atrophy/cerebral atrophy (such as especially diagnosed by magnetic resonance imaging (MRI)); wherein it is understood that such MS may notably be relapsing-remitting multiple sclerosis (RRMS), secondary progressive multiple sclerosis (SPMS), or primary progressive multiple sclerosis (PPMS).

33) A further embodiment relates to COMPOUND, or a pharmaceutically acceptable salt thereof, for use according to embodiment 22), wherein such use is for the treatment of a patient diagnosed with MS; wherein said treatment improves the symptoms of MS, wherein such improvement of symptoms of MS may especially be expressed by an effect of remyelination; wherein it is understood that such MS may notably be relapsing-remitting multiple sclerosis (RRMS), secondary progressive multiple sclerosis (SPMS), or primary progressive multiple sclerosis (PPMS).

34) A further embodiment relates to COMPOUND, or a pharmaceutically acceptable salt thereof, for use according to embodiment 22), wherein such use is for the prevention/prophylaxis of MS, wherein said prevention/prophylaxis of MS comprises delaying the onset of MS in a patient who has experienced a CIS/has been diagnosed as having experienced a CIS; wherein it is understood that such MS may notably be relapsing-remitting multiple sclerosis (RRMS).

35) A further embodiment relates to COMPOUND, or a pharmaceutically acceptable salt thereof, for use according to any one of embodiments 30) to 34), wherein said prophylaxis/prevention or treatment induces an effect of remyelination.

36) A further embodiment relates to COMPOUND, or a pharmaceutically acceptable salt thereof, for use according to any one of embodiments 16) to 35), wherein the S1P1 receptor modulator, or a pharmaceutically acceptable salt thereof, is fingolimod, ponesimod, siponimod, ozanimod, cenerimod, etrasimod, amiselimod, ceralifimod, GSK 2018682, or CS-0777; or, in addition, mocravimod (especially fingolimod, ponesimod, siponimod, or ozanimod; or, in addition, cenerimod); or a pharmaceutically acceptable salt thereof.

37) A further embodiment relates to COMPOUND, or a pharmaceutically acceptable salt thereof, for use according to any one of embodiments 16) to 35), wherein the S1P1 receptor modulator, or a pharmaceutically acceptable salt thereof, is fingolimod, ponesimod, siponimod, ozanimod, cenerimod, etrasimod, (especially fingolimod, ponesimod, siponimod, or ozanimod; or, in addition, cenerimod), or a pharmaceutically acceptable salt thereof.
In a sub-embodiment, such combination use is (especially with fingolimod, ponesimod, siponimod, or ozanimod) for the prevention or treatment of an autoimmune and/or inflammatory demyelinating disease or disorder (in particular for multiple sclerosis (MS)), or (notably with cenerimod, ozanimod or etrasimod; especially with cenerimod or etrasimod) for the prevention or treatment of an inflammatory bowel disease including Crohn's disease and ulcerative colitis; or of systemic lupus erythematosus (SLE).

38) A further embodiment relates to COMPOUND, or a pharmaceutically acceptable salt thereof, for use according to any one of embodiments 16) to 35), wherein the S1P1 receptor modulator, or a pharmaceutically acceptable salt thereof, is cenerimod, ozanimod, etrasimod, or amiselimod (especially cenerimod or etrasimod), or a pharmaceutically acceptable salt thereof.
In a sub-embodiment, such combination use is especially for the prevention or treatment of an inflammatory bowel disease especially selected from Crohn's disease and ulcerative colitis; or for the prevention or treatment of systemic lupus erythematosus (SLE).

39) A further embodiment relates to COMPOUND, or a pharmaceutically acceptable salt thereof, for use according to any one of embodiments 16) to 35), wherein the S1P1 receptor modulator, or a pharmaceutically acceptable salt thereof, is fingolimod, or a pharmaceutically acceptable salt thereof.

40) A further embodiment relates to COMPOUND, or a pharmaceutically acceptable salt thereof, for use according to any one of embodiments 16) to 35), wherein the S1P1 receptor modulator, or a pharmaceutically acceptable salt thereof, is ponesimod, or a pharmaceutically acceptable salt thereof.

41) A further embodiment relates to COMPOUND, or a pharmaceutically acceptable salt thereof, for use according to any one of embodiments 16) to 35), wherein the S1P1 receptor modulator, or a pharmaceutically acceptable salt thereof, is siponimod, or a pharmaceutically acceptable salt thereof.

42) A further embodiment relates to COMPOUND, or a pharmaceutically acceptable salt thereof, for use according to any one of embodiments 16) to 35), wherein the S1P1 receptor modulator, or a pharmaceutically acceptable salt thereof, is ozanimod, or a pharmaceutically acceptable salt thereof.

43) A further embodiment relates to COMPOUND, or a pharmaceutically acceptable salt thereof, for use according to any one of embodiments 16) to 35), wherein the S1P1 receptor modulator, or a pharmaceutically acceptable salt thereof, is cenerimod, or a pharmaceutically acceptable salt thereof.
In a sub-embodiment, such combination use is especially for the prophylaxis/prevention or treatment of an autoimmune and/or inflammatory demyelinating disease or disorder according to any one of embodiments 22) to 29); especially for the prophylaxis/prevention or treatment of MS according to any one of embodiments 30) to 35).
In another sub-embodiment, such combination use is especially for the prophylaxis/prevention or treatment of systemic lupus erythematosus (SLE).

44) A further embodiment relates to COMPOUND, or a pharmaceutically acceptable salt thereof, for use according to any one of embodiments 16) to 35), wherein the S1P1 receptor modulator, or a pharmaceutically acceptable salt thereof, is etrasimod, or a pharmaceutically acceptable salt thereof.

45) A further embodiment relates to COMPOUND, or a pharmaceutically acceptable salt thereof, for use according to any one of embodiments 16) to 44); wherein said S1P1 receptor modulator, or a pharmaceutically acceptable salt thereof, is to be administered in a pharmaceutical dosage form suitable for the oral administration of said S1P1 receptor modulator, wherein
fingolimod, or a pharmaceutically acceptable salt thereof, if present, is to be administered in said pharmaceutical dosage form in a unit dose suitable for the oral administration of a total of about 0.5 mg or below per day of fingolimod;
siponimod, or a pharmaceutically acceptable salt thereof, if present, is to be administered in said pharmaceutical dosage form in a unit dose suitable for the oral administration of a total of about 2 mg or below per day of siponimod;
ponesimod, or a pharmaceutically acceptable salt thereof, if present, is to be administered in said pharmaceutical dosage form in a unit dose suitable for the oral administration of a total of about 20 mg or below per day (especially about 10-20 mg per day, in particular 20 mg per day, or 10 mg per day) of ponesimod; and
ozanimod, or a pharmaceutically acceptable salt thereof, if present, is to be administered in said pharmaceutical dosage form in a unit dose suitable for the oral administration of a total of about 1 mg or below per day (especially about 0.5-1 mg per day, in particular 1 mg per day, or 0.5 mg per day) of ozanimod;
cenerimod, or a pharmaceutically acceptable salt thereof, if present, is to be administered in said pharmaceutical dosage form in a unit dose suitable for the oral administration of a total of about 4 mg or below per day (especially about 2-4 mg per day, in particular 4 mg per day, or 2 mg per day) of cenerimod;
etrasimod, or a pharmaceutically acceptable salt thereof, if present, is to be administered in said pharmaceutical dosage form in a unit dose suitable for the oral administration of a total of about 2 mg or below per day (especially about 1-2 mg per day, in particular 2 mg per day, or 1 mg per day) of etrasimod;
amiselimod, or a pharmaceutically acceptable salt thereof, if present, is to be administered in said pharmaceutical dosage form in a unit dose suitable for the oral administration of a total of about 0.4 mg or below per day (especially about 0.2-0.4 mg per day, in particular 0.4 mg per day, or 0.2 mg per day) of amiselimod.

The above dosage forms are especially intended for once daily (qd) dosing of said unit dose.

46) A further embodiment relates to COMPOUND, or a pharmaceutically acceptable salt thereof, for use according to any one of embodiments 16) to 45); wherein said S1P1 receptor modulator, or a pharmaceutically acceptable salt thereof, is to be administered in a pharmaceutical dosage form suitable for the oral administration of said S1P1 receptor modulator, wherein
fingolimod, or a pharmaceutically acceptable salt thereof, if present, is to be administered in said pharmaceutical dosage form in a unit dose suitable for the oral administration of a total of about 0.5 mg or below per day of fingolimod;
siponimod, or a pharmaceutically acceptable salt thereof, if present, is to be administered in said pharmaceutical dosage form in a unit dose suitable for the oral administration of a total of about 2 mg or below per day of siponimod;
ponesimod, or a pharmaceutically acceptable salt thereof, if present, is to be administered in said pharmaceutical dosage form in a unit dose suitable for the oral administration of a total of about 10 mg or below per day of ponesimod; and
ozanimod, or a pharmaceutically acceptable salt thereof, if present, is to be administered in said pharmaceutical dosage form in a unit dose suitable for the oral administration of a total of about 0.5 mg or below per day of ozanimod;
cenerimod, or a pharmaceutically acceptable salt thereof, if present, is to be administered in said pharmaceutical dosage form in a unit dose suitable for the oral administration of a total of about 2 mg or below per day of cenerimod;
etrasimod, or a pharmaceutically acceptable salt thereof, if present, is to be administered in said pharmaceutical dosage form in a unit dose suitable for the oral administration of a total of about 1 mg or below per day of etrasimod;
amiselimod, or a pharmaceutically acceptable salt thereof, if present, is to be administered in said pharmaceutical dosage form in a unit dose suitable for the oral administration of a total of about 0.2 mg or below per day of amiselimod.

The above dosage forms are especially intended for once daily (qd) dosing of said unit dose.

47) A further embodiment relates to COMPOUND, or a pharmaceutically acceptable salt thereof, for use according to any one of embodiments 16) to 46), wherein said S1P1 receptor modulator, or a pharmaceutically acceptable salt thereof, is to be administered in a dose which is a tolerated efficacious dose when given as a single therapy (e.g. as indicated in an approval letter for such S1P1 receptor modulator for the respective disease or disorder when given as a single therapy), or in a dose which is lower than such tolerated efficacious dose when given as a single therapy.

48) A further embodiment relates to COMPOUND, or a pharmaceutically acceptable salt thereof, for use according to any one of embodiments 16) to 46), wherein said S1P1 receptor modulator, or a pharmaceutically acceptable salt thereof, is to be administered in a dose which is lower than a tolerated efficacious dose when given as a single therapy (e.g. as indicated in an approval letter for such S1P1 receptor modulator for the respective disease or disorder when given as a single therapy).

49) A further embodiment relates to COMPOUND, or a pharmaceutically acceptable salt thereof, for combination use according to any one of embodiments 16) to 48), wherein COMPOUND is (to be) administered in a total dose of between about 20 mg to about 300 mg (notably about 20 mg to about 200 mg; especially about 50 mg to about 150 mg) per day of COMPOUND. In a sub-embodiment, said total dose is given/administered especially in one unit dose per day (qd; for example between about 20 mg qd to about 300 mg qd (notably about 20 mg qd to about 200 mg qd; especially about 50 mg qd to about 150 mg qd)), or in two separate unit doses per day (bid; for example between about 10 mg bid to about 150 mg bid (notably about 10 mg bid to about 100 mg bid; especially about 25 mg bid to about 75 mg bid)).

In a sub-embodiment of this embodiment 49), the particular characteristics of embodiments ii) to v) herein above apply mutatis mutandis.

50) A further embodiment, thus, relates to COMPOUND, or a pharmaceutically acceptable salt thereof, for combination use according to any one of embodiments 16) to 48), wherein COMPOUND is (to be) administered in a total dose of about 20 mg to 200 mg per day of COMPOUND; notably about 30 mg to 150 mg per day, about 40 mg to 150 mg per day, about 50 mg to 200 mg, about 50 mg to 150 mg, about 50 mg to 100 mg, or about 100 mg to 200 mg per day of COMPOUND; especially about 75 mg to 150 mg per day of COMPOUND.

In a sub-embodiment, said total dose is given/administered especially in one unit dose per day (qd), or in two separate unit doses per day (bid).

Accordingly, COMPOUND or a pharmaceutically acceptable salt thereof according to this invention is especially for use in combination (or co-therapy) with said further pharmaceutically active ingredients.

A combined treatment (or co-therapy) may be effected simultaneously (in a fixed dose or in a non-fixed dose), separately, or over a period of time (especially simultaneously).

“Simultaneously”, when referring to an administration type, means in the present application that the administration type concerned consists in the administration of two or more active ingredients and/or treatments at approximately the same time; wherein it is understood that a simultaneous administration will lead to exposure of the subject to the two or more active ingredients and/or treatments at the same time. In this context, the term “the same time” refers notably to a dosing regimen/periodicity which is essentially daily for all active ingredients, i.e. the administration of said two or more active ingredients and/or treatments occurs the same day, especially at least once at about the same time of said day.

When administered simultaneously, said two or more active ingredients may be administered:
in a fixed dose combination, or
in a non-fixed dose combination that is equivalent to a fixed dose combination (e.g. by using two or more different pharmaceutical compositions to be administered, preferably by the same route of administration, at approximately the same time), wherein in particular said non-fixed dose combination is to be administered in the same dosing regimen/periodicity (e.g. all active ingredients are to be administered especially once per day (qd), or are to be administered twice daily (bid), or the like), or
in a non-fixed dose combination using two or more different routes of administration or dosing regimen/periodicity (e.g. one or more of the active ingredients is/are to be administered once per day (qd), whereas at least one other active ingredient is to be administered, preferably by the same route of administration, in a different dosing regimen such as twice daily (bid), or three times daily (tid), or every other day);
   wherein said co-administration leads to essentially simultaneous exposure of the subject to a pharmaceutically effective amount of the two or more active ingredients and/or treatments. An example of simultaneous administration of a non-fixed dose combination using two different pharmaceutical compositions to be administered, preferably by the same route of administration, at approximately the same time is a non-fixed dose combination wherein COMPOUND is (to be) administered once a day, and the respective S1P1 receptor modulator is (to be) administered once a day. An example of simultaneous administration of a non-fixed dose combination using two different routes of administration or dosing regimen/periodicity is a non-fixed dose combination wherein COMPOUND is (to be) administered twice a day, and the respective S1P1 receptor modulator is (to be) administered once a day. Another example would be wherein COMPOUND is (to be) administered once or twice a day, and the respective S1P1 receptor modulator is (to be) administered every other day (wherein it is understood that such co-administration will lead to the subject being exposed to a pharmaceutically effective amount of COMPOUND and of said S1P1 receptor modulator simultaneously all the time). When used in combination with an S1P1 receptor modulator the COMPOUND would especially be used “simultaneously”.

“Fixed dose combination”, when referring to an administration type, means in the present application that the administration type concerned consists in the administration of one single pharmaceutical composition comprising the two or more active ingredients, such as especially the pharmaceutical compositions of any one of embodiments 1) to 15).

“Separately”, when referring to an administration type, means in the present application that the administration type concerned consists in the administration of two or more active ingredients and/or treatments at different points in time; wherein it is understood that a separate administration will lead to a treatment phase (e.g. at least 1 h, notably at least 6 h, especially at least 12 h) where the subject is exposed to the two or more active ingredients and/or treatments at the same time; but a separate administration may also lead to a treatment phase where for a certain period of time (e.g. at least 12 h, especially at least one day) the subject is exposed to only one of the two or more active ingredients and/or treatments. Separate administration especially refers to situations wherein at least one of the active ingredients and/or treatments is given with a periodicity substantially different from daily (such as once or twice daily) administration (e.g. wherein one active ingredient and/or treatment is given e.g. once or twice a day, and another is given e.g. once a week or at even longer distances).

By administration “over a period of time” is meant in the present application the subsequent administration of two or more active ingredients and/or treatments at different times. The term in particular refers to an administration method according to which the entire administration of one of the active ingredients and/or treatments is completed before the administration of the other/the others begins. In this way it is possible to administer one of the active ingredients and/or treatments for several months before administering the other active ingredient(s) and/or treatment(s).

The term “pharmaceutically effective amount” or “pharmaceutically efficacious amount” is to be understood as at least the minimal amount of the respective active ingredient which will lead to a pharmacological response in a subject (minimum pharmacologically effective amount). A pharmacological response can for example be assumed in case a given active ingredient is present (at some point in time during treatment such as for example at Tₘₐₓ or at trough; for active ingredients intended for chronic administration especially during the whole treatment period (e.g. including at trough)) in a concentration that (in case of an antagonist) blocks at least 20% (especially at least 50%) of a given biological target. A pharmacological response further can be assumed in case a biomarker responsive to such blockade of a given biological target is (significantly) increased/decreased compared to untreated reference (such as baseline or placebo); wherein such increase/decrease may be observed at some point in time during treatment such as for example at Tₘₐₓ or at trough; for active ingredients intended for chronic administration especially during the whole treatment period (e.g. including at trough). Preferably a pharmaceutically effective amount is within the therapeutic dosage range of an active ingredient, such range generally being defined by the range between the minimum effective dose (MED) and the maximum tolerated dose (MTD).

The term “subject” as used herein refers to a mammal, especially a human; notably to a patient, especially a human patient. Preferably,
the term refers to a (human) subject who is at risk/has been diagnosed as being at risk of developing a certain disease or disorder, and therefore is in need of prophylaxis/prevention of such disease or disorder;
or the term refers to a (human) patient who has been diagnosed with/as having a certain disease or disorder, and therefore is in need of treatment of such disease or disorder.

It is understood that any embodiment relating to COMPOUND, or a pharmaceutically acceptable salt thereof, for use in the prophylaxis/prevention or treatment of a certain disease and disorder where both CXCR7 expression or its ligands and S1P play a role as specifically defined herein, wherein COMPOUND is (intended) to be administered in combination with an S1P1 receptor modulator (especially an S1P1 receptor modulator as specifically defined in such embodiment) also relates
to such S1P1 receptor modulator, or a pharmaceutically acceptable salt thereof, for use in the prophylaxis/prevention or treatment of said disease and disorder where both CXCR7 expression or its ligands and S1P play a role; wherein said S1P1 receptor modulator is (intended) to be administered in combination with COMPOUND, or a pharmaceutically acceptable salt thereof;
to the use of COMPOUND, or of a pharmaceutically acceptable salt thereof, for the manufacture of a medicament/a pharmaceutical composition comprising COMPOUND, or a pharmaceutically acceptable salt thereof, and such S1P1 receptor modulator, or a pharmaceutically acceptable salt thereof, for use in the prophylaxis/prevention or treatment of said disease and disorder where both CXCR7 expression or its ligands and S1P play a role;
to the use of COMPOUND, or of a pharmaceutically acceptable salt thereof, for the manufacture of a medicament/pharmaceutical composition comprising, as active ingredient, COMPOUND, or a pharmaceutically acceptable salt thereof, for use in the prophylaxis/prevention or treatment of said disease and disorder where both CXCR7 expression or its ligands and S1P play a role; wherein said medicament/pharmaceutical composition is (intended) to be used in combination with such S1P1 receptor modulator, or a pharmaceutically acceptable salt thereof;
to the use of such S1P1 receptor modulator, or a pharmaceutically acceptable salt thereof, for the manufacture of a medicament/pharmaceutical composition comprising, as active ingredient, said S1P1 receptor modulator, or a pharmaceutically acceptable salt thereof, for use in the prophylaxis/prevention or treatment of said disease and disorder where both CXCR7 expression or its ligands and S1P play a role; wherein said medicament/pharmaceutical composition is (intended) to be used in combination with COMPOUND, or a pharmaceutically acceptable salt thereof;
to the use of a pharmaceutical composition comprising COMPOUND, or a pharmaceutically acceptable salt thereof, and such S1P1 receptor modulator, or a pharmaceutically acceptable salt thereof, for the prophylaxis/prevention or treatment of said disease and disorder where both CXCR7 expression or its ligands and S1P play a role;
to a medicament for the prophylaxis/prevention or treatment said disease and disorder where both CXCR7 expression or its ligands and S1P play a role, said medicament comprising COMPOUND, or a pharmaceutically acceptable salt thereof; wherein said medicament is (intended) to be administered in combination with said S1P1 receptor modulator, or a pharmaceutically acceptable salt thereof;
to a method of prophylaxis of/preventing or treating said disease and disorder where both CXCR7 expression or its ligands and S1P play a role comprising administering to a subject (preferably a human) in need thereof an effective amount of COMPOUND, or a pharmaceutically acceptable salt thereof, wherein COMPOUND is administered in combination with an effective amount of such S1P1 receptor modulator, or of a pharmaceutically acceptable salt thereof;
to a method of prophylaxis of/preventing or treating said disease and disorder where both CXCR7 expression or its ligands and S1P play a role comprising administering to a subject in need thereof an effective amount of a pharmaceutical composition comprising COMPOUND, or a pharmaceutically acceptable salt thereof, and such S1P1 receptor modulator, or a pharmaceutically acceptable salt thereof; and
to a method of prophylaxis of/preventing or treating said disease and disorder where both CXCR7 expression or its ligands and S1P play a role comprising administering to a subject (preferably a human) in need thereof an effective amount of such S1P1 receptor modulator, or of a pharmaceutically acceptable salt thereof, wherein said S1P1 receptor modulator is administered in combination with an effective amount of COMPOUND, or of a pharmaceutically acceptable salt thereof.

Likewise, any embodiment relating to COMPOUND, or a pharmaceutically acceptable salt thereof, for use in the prophylaxis/prevention or treatment of a certain disease and disorder where CXCR7 expression or its ligands play a role is to be understood as also referring to the use of COMPOUND, or of a pharmaceutically acceptable salt thereof, in the prophylaxis/prevention or treatment of said certain disease and disorder; and to a method of prophylaxis of/preventing or treating said certain disease and disorder, said method comprising administering to a subject in need thereof an effective amount of a pharmaceutical composition comprising COMPOUND, or a pharmaceutically acceptable salt thereof.

Where the plural form is used for compounds, salts, pharmaceutical compositions, diseases and the like, this is intended to mean also a single compound, salt, or the like.

Definitions provided herein are intended to apply uniformly to the compositions as defined in any one of embodiments 1) to 50), and, mutatis mutandis, throughout the description and the claims unless an otherwise expressly set out definition provides a broader or narrower definition. It is well understood that a definition or preferred definition of a term defines and may replace the respective term independently of (and in combination with) any definition or preferred definition of any or all other terms as defined herein.

Any reference to compounds is to be understood as referring also to the salts (and especially the pharmaceutically acceptable salts) of such compounds, as appropriate and expedient.

The term “pharmaceutically acceptable salts” refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example “Handbook of Pharmaceutical Salts. Properties, Selection and Use.”, P. Heinrich Stahl, Camille G. Wermuth (Eds.), Wiley-VCH, 2008; and “Pharmaceutical Salts and Co-crystals”, Johan Wouters and Luc Quere (Eds.), RSC Publishing, 2012.

The term “consisting essentially of” is understood in the context of the present invention to mean especially that the respective composition consists in an amount of at least 90, notably of at least 95, especially of at least 99, and preferably in an amount of 100 percent by weight (i.e. in the meaning of “consisting of”) of the respective composition in the amounts as explicitly stated in the respective embodiment. The term “comprising” is preferably to be understood in the meaning of the term “consisting essentially of”.

The term “essentially”, is understood in the context of the present invention to mean especially that the respective amount/purity/time etc. is at least 90, especially at least 95, and notably at least 99 percent of the respective total.

For example when used in the term “essentially simultaneous exposure” is understood to mean especially that the respective exposure results in simultaneous exposure of pharmaceutically effective amounts of all combination active ingredients during at least 90, especially at least 95, and notably at least 99 percent of the time, i.e. of the day in case chronic/steady state exposure to the pharmaceutically active ingredients is contemplated.

For example when used in a term such as “essentially pure” is understood in the context of the present invention to mean especially that the respective composition/compound etc. consists in an amount of at least 95, and notably of at least 99 percent by weight of the respective pure composition/compound/crystalline form etc.

The term “enantiomerically enriched” is understood in the context of the present invention to mean especially that at least 90, preferably at least 95, and most preferably at least 99 percent by weight of the COMPOUND are present in form of one enantiomer of the COMPOUND. It is understood that COMPOUND is present in enantiomerically enriched absolute (3S,4S)-configuration, preferably in essentially pure absolute (3S,4S)-configuration.

For avoidance of any doubt, it is well understood that any pharmaceutical composition comprising COMPOUND in a pharmaceutically effective amount may additionally comprise further conventional excipients and/or additives, which may be used alone or in combination (quantum satis, i.e. wherein the maximum amounts of said further conventional ingredients and/or additives may need to be reduced to make up the total ww % of 100). It is understood that the total amount expressed in “ww %” of a certain composition is 100. The expression “ww %” (or % (w/w)) refers to a percentage by weight compared to the total weight of the composition considered.

The production of pharmaceutical compositions according to this invention can be effected in a manner which will be familiar to any person skilled in the art (see for example R. C. Rowe, P. J. Seskey, S. C. Owen, Handbook of Pharmaceutical Excipients, 5th edition, Pharmaceutical Press 2006; Remington, The Science and Practice of Pharmacy, 21st Edition (2005), Part 5, “Pharmaceutical Manufacturing” [published by Lippincott Williams & Wilkins]) by bringing the combination active ingredients of the present invention, optionally in combination with other therapeutically valuable substances, into a galenical administration form together with suitable, non-toxic, inert, pharmaceutically acceptable solid or liquid carrier materials and, if desired, usual pharmaceutical adjuvants. A pharmaceutical composition for oral administration may in particular be in form of a capsule or in form of a tablet.

Dosage forms suitable for enteral administration may be tablets. Alternatively, dosage forms suitable for enteral administration may be capsules (especially a hard gelatine capsules) filled with a pharmaceutical composition comprising an efficacious amount of COMPOUND. Any type of capsule that is usually used to contain pharmaceutical compositions in the form of powder or pellets such as hard gelatine capsules, HPMC capsules, etc. may be used in the present invention. It is understood that a capsule or tablet will comprise, in addition to COMPOUND or pharmaceutically acceptable salt thereof, optionally in combination with an S1P1 receptor modulator, or a pharmaceutically acceptable salt thereof, as defined herein, and at least one pharmaceutically acceptable, inert excipient. The term “pharmaceutical composition” as used herein is interchangeable with the terms “formulation”, “composition” or “medicament”.

Unless used regarding temperatures, the term “about” placed before a numerical value “X” refers in the current application to an interval extending from X minus 10% of X to X plus 10% of X, and preferably to an interval extending from X minus 5% of X to X plus 5% of X. In the particular case of temperatures, the term “about” placed before a temperature “Y” refers in the current application to an interval extending from the temperature Y minus 10° C. to Y plus 10° C., preferably to an interval extending from Y minus 5° C. to Y plus 5° C., notably to an interval extending from Y minus 3° C. to Y plus 3° C. Room temperature means a temperature of about 25° C. When in the current application the term n equivalent(s) is used wherein n is a number, it is meant and within the scope of the current application that n is referring to about the number n, preferably n is referring to the exact number n.

Whenever the word “between” or “to” is used to describe a numerical range, it is to be understood that the end points of the indicated range are explicitly included in the range. For example: if a temperature range is described to be between 40° C. and 80° C. (or 40° C. to 80° C.), this means that the end points 40° C. and 80° C. are included in the range; or if a variable is defined as being an integer between 1 and 4 (or 1 to 4), this means that the variable is the integer 1, 2, 3, or 4.

Particular embodiments of the invention are described in the following examples, which serve to illustrate the invention in more detail without limiting its scope in any way.

### EXPERIMENTAL PROCEDURES

### Abbreviations

The following abbreviations are used throughout the specification and the examples:
b.i.d. (bis in die): also bid; twice daily
CFA complete Freund's adjuvant
CPZ cuprizone
EAE experimental autoimmune encephalomyelitis
Fig. Figure
h hour(s)
IBD Inflammatory Bowel Disease
MOG myelin oligodendrocyte glycoprotein
MS multiple sclerosis
NFL neurofilament light chain
ns non significant
PLP proteolipid protein
q.d. (quaque die): also qd; once daily
SEM standard error of the mean
   Examples of Therapeutic Uses of COMPOUND as a Monotherapy or in Combination with an S1P1 Receptor Modulator

Therapeutic effects can be modeled in multiple animal models indicative of diseases and disorders where both CXCR7 expression or its ligands and S1P play a role.

### Example A

The efficacy of COMPOUND and fingolimod alone or in combination can be determined in a mouse model of inflammatory demyelinating diseases, relevant for example for multiple sclerosis (MS).

CXCL12 or CXCL11 plasma concentrations may be determined using methods well known in the art, e.g. an Ella® immunoassay (Bio-Techne®), or a commercial quantikine ELISA mouse CXCL12/SDF1α kit from R&D systems.

### a) Dose Finding Experiments in Naïve Mice:

In a pilot experiment, healthy female C57BL/6 mice are dosed orally for 3 days with COMPOUND at 30 and 100 mg/kg, twice daily (b.i.d.). The goal is to assess the dose-effect relationship of COMPOUND on CXCL12 elevation in plasma (which is an established biomarker to monitor pharmacological activity of a CXCR7 receptor antagonist) in this strain of mouse over 24 h.

Based on this experiment only the highest dose of COMPOUND tested provides COMPOUND plasma exposure at trough (14 hours after last dosing) to sustain plasma CXCL12 increase over 24 h, in naïve C57BL/6 mice (dose 100 mg/kg, b.i.d.).

In parallel to the measurement of target engagement of COMPOUND, a second pilot experiment is performed. Healthy female C57BL/6 receive fingolimod at different doses in the range from 0.01 to 0.3 mg/kg, once daily (q.d.). The goal is to assess the dose-effect relationship of fingolimod on lymphocyte count in peripheral blood (which is an established biomarker to monitor pharmacological activity of S1P1 receptor modulators) in this strain of mouse over 24 h. Based on this experiment, the selected dose of fingolimod for combination experiment shows partial efficacy on lymphocyte count reduction over 24 h (dose 0.03 mg/kg, q.d.).

### b) Monotherapy Efficacy Experiment in Myelin Oligodendrocyte Glycoprotein (MOG)-Induced Experimental Autoimmune Encephalomyelitis (EAE) Model:

The efficacy of COMPOUND in myelin oligodendrocyte glycoprotein (MOG)-induced experimental autoimmune encephalomyelitis (EAE) can be determined in a pilot experiment. The goal is to assess the dose-effect relationship of COMPOUND on efficacy and on plasma CXCL12 increase, in a mouse model of MS.

Female C57BL/6 mice are immunized with an emulsion of MOG in complete Freund's adjuvant (CFA) and pertussis toxin (day 0). A total of 150 μg of MOG is injected per mice at two sites subcutaneously in each flank of the abdomen. Mice are injected intraperitoneally with pertussis toxin a second time, 2 days after the first injection (day 2). Within 9 to 14 days, mice develop signs of paralysis which will be graded on a scale from 0 to 5 assessing the tail and limbs defined as: 0=no clinical sign of EAE, 0.5=end tail paralyzed or one limb weakness, 1=tail paralyzed or two limbs weakness, 1.5=tail paralyzed and one hind limb weakness, 2=tail paralyzed and bilateral partial hind limb paralysis, 2.5=tail paralyzed and unilateral complete hind limb paralysis, 2.75=score of 2.5+unilateral partial hind limb paralysis, 3=complete bilateral hind limb paralysis, 3.25=3+unilateral partial forelimb paralysis, 3.5=3+unilateral complete forelimb paralysis, 4=complete paralysis (moribund) and, 5=death or euthanized.

The disease progression follows a chronic progressive course with no remission.

Groups of 9 to 10 mice are dosed orally starting at the day of disease induction (day 0) with different doses of COMPOUND. It consists of four treatment groups:
1. Vehicle (water) b.i.d., from day 0
2. COMPOUND (10 mg/kg) b.i.d., from day 0
3. COMPOUND (30 mg/kg) b.i.d., from day 0
4. COMPOUND (100 mg/kg) b.i.d., from day 0

Clinical scores are assessed on a daily basis and disease development is compared between vehicle-treated mice and mice receiving COMPOUND. The cumulative disease score is calculated for each mouse by summing all the daily clinical scores over the 29-days study period. The experiment is terminated at day 29, 1 to 4 hours post dosing. Plasma samples are taken for COMPOUND concentration determination and for the measurement of biomarker for CXCR7 target engagement (CXCL12 levels). CXCL12 plasma concentrations are determined using a commercial quantikine ELISA mouse CXCL12/SDF1c kit from R&D systems, according to manufacturer's instructions.

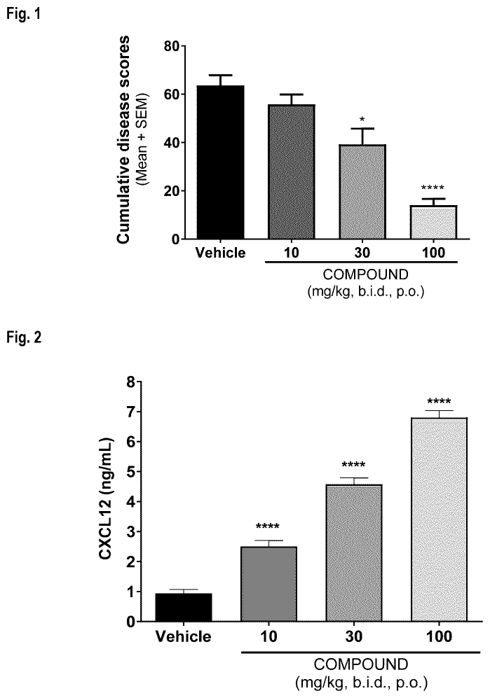

The results from the efficacy experiment are shown in FIG. 1-2. COMPOUND administered in a preventive setting, exhibits dose-dependent efficacy on the overall extent of EAE disease as shown by a reduction in the mean cumulative disease scores over the 29-days study (FIG. 1). Efficacy is associated with a dose-dependent increase of plasma CXCL12 levels (FIG. 2).

Based on the pilot experiments described above one dose of COMPOUND is selected for the combination efficacy experiment. The dose selected provides COMPOUND plasma exposure at trough to sustain CXCL12 plasma levels over 24 hours and significantly reduces overall burden of EAE disease when given in a preventive setting (dose: 100 mg/kg, b.i.d.).

In parallel to the measurement of dose-dependent efficacy of COMPOUND in the EAE model, a second pilot experiment to assess the efficacy of the selected dose of Fingolimod is performed.

Groups of 10 mice are dosed orally starting at the day of disease induction (day 0). It consists of two treatment groups:
1. Vehicle (0.5% Methylcellulose/0.5% Tween® 80) b.i.d., from day 0
2. Fingolimod (0.03 mg/kg) q.d.+Vehicle q.d., from day 0

Clinical scores are assessed on a daily basis and disease development is compared between vehicle-treated mice and mice receiving fingolimod. The cumulative disease score is calculated for each mouse by summing all the daily clinical scores over the 27-days study period. The experiment is terminated at day 27.

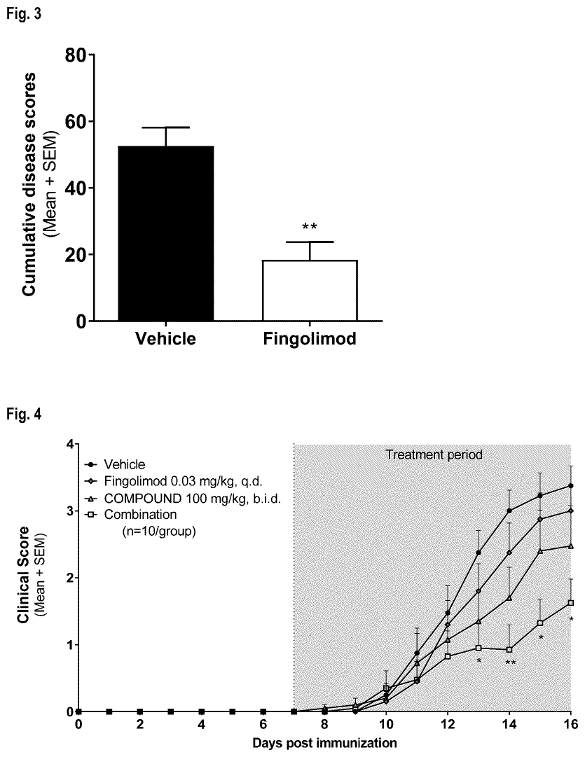

Fingolimod administered in a preventive setting at the selected dose (0.03 mg/kg), exhibits efficacy on the overall extent of EAE disease as shown by a reduction in cumulative disease scores over the 27-days study (FIG. 3). Efficacy of Fingolimod was not associated with an increase of plasma CXCL12 levels.

### c) Combination Efficacy Experiment in the MOG-Induced EAE Model:

The combination efficacy experiment is performed in the same mouse EAE model as described for the pilot experiments.

Groups of 10 mice are dosed orally starting just before the onset of disease (day 7). It consists of four treatment groups:
1. Vehicle (0.5% Methylcellulose/0.5% Tween® 80) b.i.d., from day 7
2. Fingolimod (0.03 mg/kg) q.d.+Vehicle q.d., from day 7
3. COMPOUND (100 mg/kg) b.i.d., from day 7
4. Fingolimod (0.03 mg/kg) q.d.+COMPOUND (100 mg/kg) b.i.d., from day 7

Clinical scores are assessed on a daily basis and disease development is compared between vehicle-treated mice and mice receiving the different treatments. The experiment is terminated at day 16 or day 17. Hematology parameters, including lymphocyte count, are measured at the termination of the experiment. Plasma samples are taken for COMPOUND concentration determination and for the measurement of biomarkers for CXCR7 target engagement (CXCL12 levels) and for axonal damage (Neurofilament light chain (NFL) levels). CXCL12 plasma concentrations are determined using a commercial quantikine ELISA mouse CXCL12/SDF1c kit from R&D systems, according to manufacturer's instructions.

This experiment is suitable to show whether the addition of a dose of COMPOUND, that shows target engagement at all times and efficacy as monotherapy in a EAE model, shows added benefit to a dose of fingolimod that is only partially efficacious on lymphocyte count.

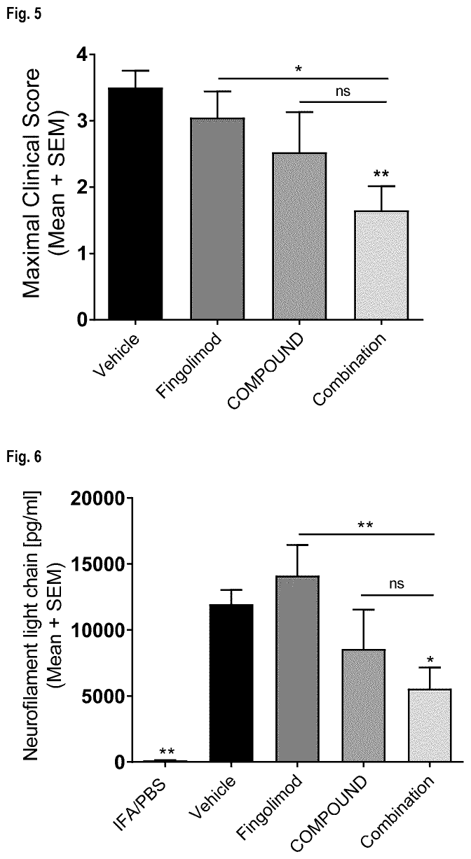

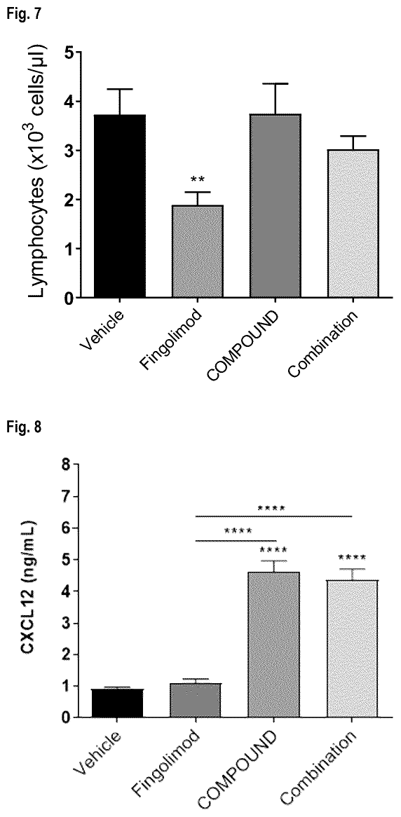

The results from the combination efficacy experiment are shown in FIG. 4-8. COMPOUND and fingolimod administered in a therapeutic setting, just before onset, exhibited moderate and minimal efficacy, respectively, on the clinical scores of the disease in the mouse EAE model (FIG. 4). When combined, the two compounds showed synergistic efficacy from day 14 onwards on the EAE disease course, reducing the severity of the disease (FIG. 4 and FIG. 5) and NFL plasma concentrations (FIG. 6); presence of NFL is indicative of irreversible/axonal damage. This synergistic effect may not be explained by an additive effect of the combination, nor on fingolimod-induced lymphocyte count reduction in peripheral blood (FIG. 7), nor on COMPOUND-induced CXCL12 plasma levels increase at the termination of the experiment (FIG. 8).

FIG. 1 shows the dose-dependent effect of COMPOUND on the overall extent of EAE disease as assessed by cumulative disease scores, defined as the sum of the clinical scores for each mouse over the 29-days study. Mice were treated from Day 0. Data are presented as mean values+SEM; n=9-10/group. *p<0.05, ****p<0.0001 vs. vehicle-treated EAE mice, using Kruskal-Wallis test, followed by Dunn's multiple comparisons test.

FIG. 2 shows the dose-dependent effect of COMPOUND on CXCL12 plasma concentration in the mouse MOG-induced EAE model. Data are presented as mean values+SEM; n=7-10/group. ****p<0.0001 vs. vehicle-treated EAE mice, using one-way ANOVA test, followed by Dunnett's multiple comparisons test.

FIG. 3 shows the effect of fingolimod (0.03 mg/kg, q.d.) on the overall extent of EAE disease as assessed by cumulative disease scores, defined as the sum of the clinical scores for each mouse over the 27-days study. Mice were treated from Day 0. Data are presented as mean values+SEM; n=10/group, ** p<0.01 vs. vehicle-treated EAE mice, using Mann Whitney test.

FIG. 4 shows the therapeutic efficacy of COMPOUND, fingolimod, and their combination on the average clinical score in the EAE mouse model. Mice were treated from Day 7 on until the end of study. Data are presented as mean values+SEM; n=10/group. *p<0.05, ** p<0.01 vs. vehicle-treated EAE mice, using Kruskal-Wallis test, followed by Dunn's multiple comparisons test.

FIG. 5 shows the therapeutic effect of COMPOUND, fingolimod, and their combination on the severity of mouse EAE disease, represented as the maximal clinical score reached over the 16 days study. Data are presented as mean values+SEM; n=10/group. *p<0.05, ** p<0.01 using Kruskal-wallis test, followed by uncorrected Dunn's multiple comparisons test.

FIG. 6 shows the therapeutic effect of COMPOUND, fingolimod, and their combination on neurofilament light chain plasma concentration in a mouse EAE model. Data are presented as mean values+SEM; n=8-9/group. *p<0.05, ** p<0.01 using one-way ANOVA test, followed by uncorrected Fisher's LSD multiple comparisons test.

FIG. 7 shows the effect of COMPOUND, fingolimod, and their combination on blood lymphocyte count in a mouse EAE model. Data are presented as mean values+SEM; n=8-10/group. **p<0.01, using one-way ANOVA test, followed by uncorrected Fisher's LSD multiple comparisons test vs Vehicle-treated EAE mice.

FIG. 8 shows the effect of COMPOUND, fingolimod, and their combination on plasma CXCL12 concentrations in a mouse EAE model. Data are presented as mean values+SEM; n=8-10/group. ****p<0.0001, using one-way ANOVA test, followed by uncorrected Fisher's LSD multiple comparisons test.

### Example B

Experiment 1): The direct effects of COMPOUND on myelination can be determined in the cuprizone-induced demyelination mouse model, where primary demyelination is not immune-mediated.

Male C57BL/6 mice are exposed to cuprizone (CPZ; 150 mg/kg, b.i.d.), a chopper chelating agent which leads to the death of mature oligodendrocytes, by oral gavage twice daily for six weeks. COMPOUND (100 mg/kg, b.i.d.) is given either in a preventive setting, i.e. co-administered with cuprizone from day 0 (COMPOUND prev-CPZ); or in a therapeutic setting, i.e. starting 3 weeks after cuprizone exposure start until the end of the experiment (COMPOUND ther-CPZ).

The experiment is terminated after 6 weeks of cuprizone exposure. Plasma samples are taken for COMPOUND concentration determination and for the measurement of biomarkers for CXCR7 target engagement (CXCL12 levels). CXCL12 plasma concentrations are measured as previously described in example A. Brain samples are isolated and fixed for histopathological and immunohistochemistry examinations to assess the degree of demyelination (Luxol Fast Blue for myelin staining) and the loss of mature oligodendrocytes (GSTiT staining).

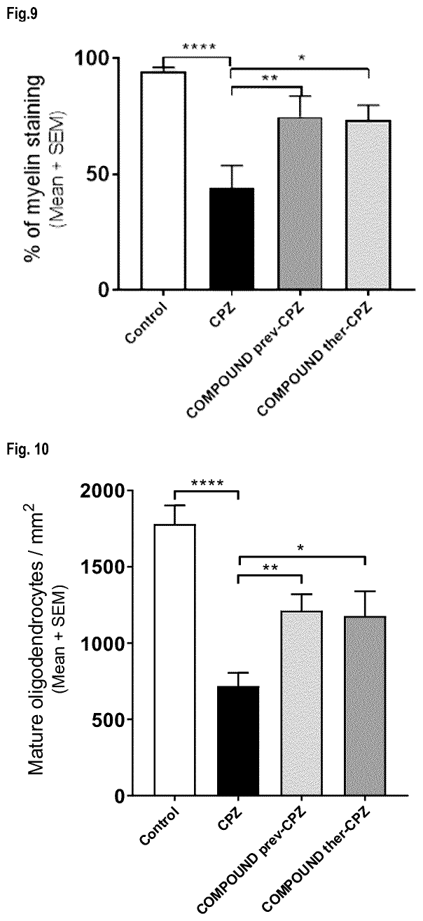

The results from this study are shown in FIG. 9-10. After 6 weeks of exposure, cuprizone induced significant demyelination (FIG. 9) and significant loss of mature oligodendrocytes (FIG. 10). While cuprizone was still administered, both COMPOUND treatment regimens (preventive: COMPOUND prev-CPZ and therapeutic: COMPOUND ther-CPZ) increased significantly the myelin stain (FIG. 9) and mature oligodendrocyte counts (FIG. 10) in the corpus callosum.

FIG. 9 shows the effect of COMPOUND co-administered with cuprizone, in the same formulation, starting from Day 0 (COMPOUND prev-CPZ) or starting after 3 weeks of cuprizone exposure (COMPOUND ther-CPZ) on demyelination in the mouse cuprizone-induced demyelination model. Rostral coronal sections were stained with luxol fast blue and intensity of the staining in the corpus callosum was quantified using Orbit image analysis software. Results are expressed as mean+SEM, n=7-8 mice per group. * p<0.05, **p<0.01, **** p<0.0001, versus vehicle-treated CPZ mice (CPZ), using a one-way ANOVA followed by uncorrected Fisher's multiple comparisons test.

FIG. 10 shows the effect of COMPOUND co-administered with cuprizone, in the same formulation, starting from Day 0 (COMPOUND prev-CPZ) or starting after 3 weeks of cuprizone exposure (COMPOUND ther-CPZ) on mature oligodendrocyte numbers in the mouse cuprizone-induced demyelination model. Rostral coronal sections were stained with GSTπ and quantitative analysis in the corpus callosum was quantified using Orbit image analysis software. Results are expressed as the mean number of cells normalized by the selected region of interest in mm²+SEM, n=6-8 mice per group. * p<0.05, **p<0.01, **** p<0.0001, versus vehicle-treated CPZ mice (CPZ), using a one-way ANOVA followed by uncorrected Fisher's multiple comparisons test.

Experiment 2): In a Second Experiment, the Therapeutic Effect of COMPOUND on Myelination is Determined in the Model of Toxic Demyelination Induced by Cuprizone, where Spontaneous Remyelination Occurs after Cuprizone Withdrawal.

Male C57BL/6 mice are exposed to a 0.2% cuprizone diet for six weeks and then switched to control food for one more week. Groups of 8-9 mice are dosed orally starting after five weeks of cuprizone exposure. It consists of three treatment groups:
1. Vehicle (0.5% Methylcellulose/0.5% Tween® 80) b.i.d., from week 5 to week 7
2. Fingolimod (0.3 mg/kg) q.d.+Vehicle q.d., from week 5 to week 7
3. COMPOUND (100 mg/kg) b.i.d., from week 5 to week 7

The experiment is terminated after two weeks of treatment (week 7), meaning one week after cuprizone withdrawal. Hematology parameters, including lymphocyte count, are measured at the termination of the experiment, one hour after the last dosing. Plasma samples are taken for COMPOUND concentration determination and for the measurement of biomarkers for CXCR7 target engagement (CXCL12 levels). Brain samples are isolated and fixed for histopathological and immunohistochemistry examinations to assess the degree of demyelination (Luxol Fast Blue for myelin staining).

This experiment is suitable to compare head to head a dose of COMPOUND—that shows target engagement at all times and efficacy as monotherapy in both the EAE model and cuprizone model—with a dose of fingolimod that is fully efficacious on lymphocyte count reduction over 24 hours (dose: 0.3 mg/kg, q.d.).

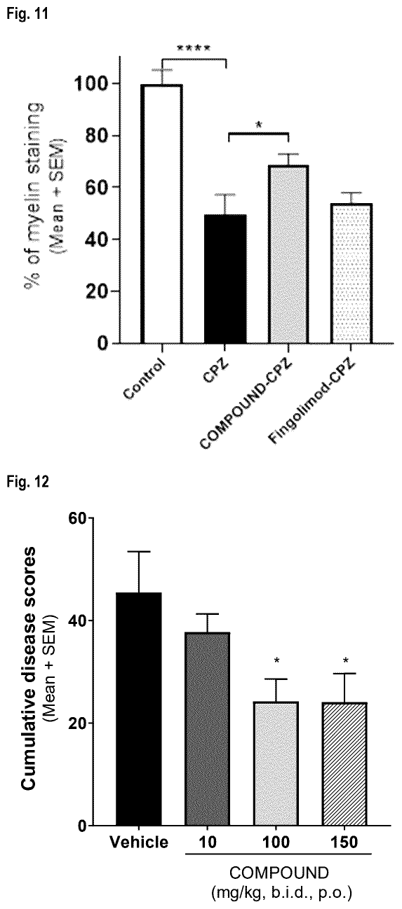

The results from this study are shown in FIG. 11. After 6 weeks of cuprizone exposure followed by one week of control food, vehicule-treated mice showed significant demyelination in the corpus callosum. One week after CPZ withdrawal, COMPOUND accelerated significantly spontaneous remyelination while fingolimod had no effect (FIG. 11).

FIG. 11 shows the therapeutic effect of COMPOUND and fingolimod starting one week before cuprizone withdrawal on demyelination/remyelination in the mouse cuprizone-induced demyelination model. Rostral coronal sections were stained with luxol fast blue and intensity of the staining in the corpus callosum was quantified using Orbit image analysis software. Results are expressed as mean+SEM, n=7-8 mice per group. * p<0.05, **** p<0.0001, versus vehicle-treated CPZ mice (CPZ), using a one-way ANOVA followed by uncorrected Fisher's multiple comparisons test.

### Example C

The therapeutic efficacy of COMPOUND and siponimod alone or in combination can be determined in a mouse model of inflammatory demyelinating disease.

### a) Dose Finding Experiments in Healthy Mice:

In a pilot experiment, healthy female SJL/J mice are dosed orally for 2 to 3 days with siponimod at different doses in the range from 0.03 to 1 mg/kg, once daily. The goal is to assess the dose-effect relationship of siponimod on lymphocyte count in peripheral blood (which is an established biomarker to monitor pharmacological activity of S1P1 receptor modulators) in this strain of mouse over 24 h. Based on this experiment, the selected dose of siponimod for combination experiment shows full efficacy on lymphocyte count reduction over 24 h (dose 0.3 mg/kg, q.d.). Combining siponimod at the dose selected with COMPOUND at 100 mg/kg, b.i.d. does not influence each other's biomarker, namely lymphopenia and plasma CXCL12 increase, respectively, nor their respective plasma pharmacokinetics.

### b) Monotherapy Efficacy Experiment in the Proteolipid Protein (PLP)-Induced EAE Model:

The therapeutic efficacy of COMPOUND in PLP-induced EAE can be determined in a pilot experiment. The goal is to assess the dose-effect relationship of COMPOUND on efficacy and on both plasma CXCL11 and CXCL12 increase.

Female SJL/J mice are immunized with an emulsion of PLP in CFA and pertussis toxin (day 0). A total of 100 μg of PLP is injected per mice at two sites subcutaneously in each flank of the abdomen. Mice are injected intraperitoneally with pertussis toxin a second time, 2 days after the first injection (day 2). Within 9 to 16 days, mice develop signs of paralysis which will be graded on a scale from 0 to 5 assessing the tail and limbs defined as: 0=no clinical sign of EAE, 0.5=end tail paralyzed or one limb weakness, 1=tail paralyzed or two limbs weakness, 1.5=tail paralyzed and one hind limb weakness, 2=tail paralyzed and bilateral partial hind limb paralysis, 2.5=tail paralyzed and unilateral complete hind limb paralysis, 2.75=score of 2.5+unilateral partial hind limb paralysis, 3=complete bilateral hind limb paralysis, 3.25=3+unilateral partial forelimb paralysis, 3.5=3+unilateral complete forelimb paralysis, 4=complete paralysis (moribund) and, 5=death or euthanized. The disease progression follows a relapsing-remitting course with a first peak of disease occurring three to five days after the onset, followed by a remission phase and a second peak of the disease between 20-30 days after EAE induction.

Groups of 14 to 16 mice are dosed orally with different doses of COMPOUND, starting from the first signs of disease for each mouse. The study consists of four treatment groups:
1. Vehicle (water) b.i.d., from disease onset for each mouse
2. COMPOUND (10 mg/kg) b.i.d., from disease onset for each mouse
3. COMPOUND (100 mg/kg) b.i.d., from disease onset for each mouse
4. COMPOUND (150 mg/kg) b.i.d., from disease onset for each mouse

Clinical scores are assessed on a daily basis, in a blinded manner and disease development is compared between vehicle-treated mice and mice receiving COMPOUND. The cumulative disease score is calculated for each mouse by summing all the daily clinical scores over the 30-days after treatment initiation. The experiment is terminated based on the mouse enrollment day, after 30 to 33 days of COMPOUND treatment, meaning between 42 and 48 days after EAE induction. Plasma samples are taken for COMPOUND concentration determination and for the measurement of biomarkers for CXCR7 target engagement (CXCL11 and CXCL12 levels). CXCL12 plasma concentrations are determined as previously described in example A.

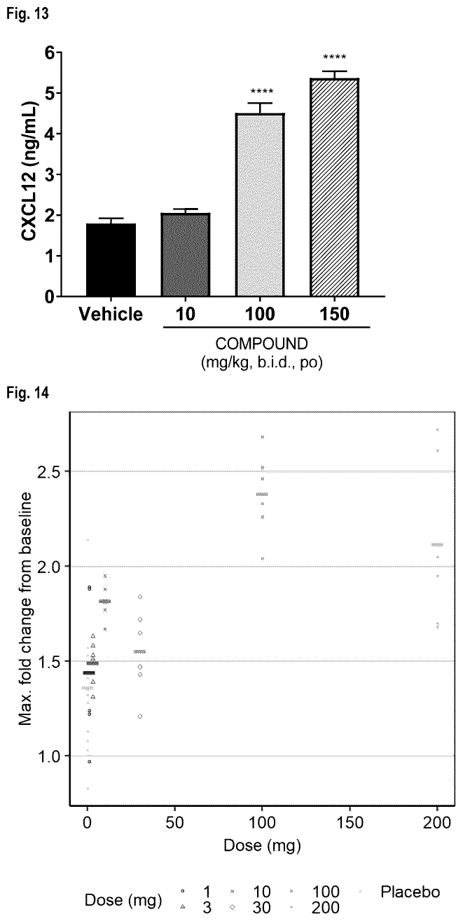

The results from the efficacy experiment are shown in FIG. 12. COMPOUND administered in a therapeutic setting, exhibits dose-dependent efficacy on the overall extent of the disease, as shown by a significant reduction in the mean cumulative disease scores (FIG. 12). Efficacy is associated with a dose-dependent increase of plasma CXCL11 and CXCL12 levels (FIG. 13).

FIG. 12 shows the dose-dependent effect of COMPOUND on the overall extent of EAE disease as assessed by cumulative disease scores, defined as the sum of clinical scores for each mouse over the 30-days after treatment initiation. Mice were treated individually starting at the first signs of disease. Data are presented as mean values+SEM; n=14-16/group. *p<0.05 vs. vehicle-treated EAE mice, using Kruskal-Wallis test, followed by uncorrected Dunn's multiple comparisons test.

FIG. 13 shows the dose-dependent effect of COMPOUND on plasma CXCL12 concentrations in the mouse PLP-induced EAE model. Data are presented as mean values+SEM; n=13-14/group. ****p<0.0001, using one-way ANOVA test, followed by uncorrected Fisher's LSD multiple comparisons test.

Based on the pilot experiment described above, one dose of COMPOUND (dose: 100 mg/kg, b.i.d.) is selected for the combination efficacy experiment. The dose selected increases CXCL11 and CXCL12 plasma levels at trough and maximally reduces clinical scores in the PLP-induced EAE model.

### c) Combination Efficacy Experiment in the PLP-Induced EAE Model:

The combination efficacy experiment is performed in the same mouse EAE model as described for the pilot experiments.

Groups of 10-15 mice are dosed orally starting at the onset of disease for each mouse. The study consists of four treatment groups:
1. Vehicle (0.5% Methylcellulose/0.5% Tween® 80) b.i.d., from EAE onset
2. Siponimod (0.3 mg/kg) q.d.+Vehicle q.d., from EAE onset
3. COMPOUND (100 mg/kg) b.i.d., from EAE onset
4. Siponimod (0.3 mg/kg) q.d.+COMPOUND (100 mg/kg) b.i.d., from EAE onset

Clinical scores are assessed on a daily basis, in a blinded manner and disease development is compared between vehicle-treated mice and mice receiving the different treatments and between combination and monotherapy treatments. In parallel, body weight of the mice are recorded daily to follow general health. The experiment is terminated after at least 30 days of COMPOUND treatment for each mouse. S1P1 and CXCR7 target engagement, namely lymphocyte count and CXCL11 and CXCL12 plasma levels, respectively, are measured at the termination of the experiment. Plasma samples are also taken for COMPOUND and siponimod concentration determination.

### Example D: The Investigation of Safety, Tolerability, Pharmacokinetics and Pharmacodynamics of COMPOUND can be Determined after Single Doses in Healthy Male Subjects

### A) Study Design (ClinicalTrials.Gov: NCT03869320)

In a randomized, double-blind, placebo-controlled first-in-human study six dose levels of COMPOUND are investigated, namely 1, 3, 10, 30, 100, and 200 mg. In each dose level group, six healthy male subjects receive COMPOUND and two healthy male subjects receive matching placebo, orally, in the fasted condition in the morning. After each dosing the subjects are monitored for 14 days to investigate (i) tolerability and safety (adverse events, vital signs, clinical laboratory, ECG), (ii) pharmacokinetics (COMPOUND concentration in plasma), and (iii) pharmacodynamics (plasma CXCL11 and CXCL12). In the 30 mg dose level group the effect of food is additionally investigated by administering the same treatment to the same subjects after a standardized high-fat breakfast. The mass balance and ADME are additionally investigated by administration of an oral COMPOUND microtracer or matching placebo in conjunction with the assigned treatment in the 100 mg dose level group. The absolute bioavailability is additionally investigated by administration of an i.v. COMPOUND microtracer or matching placebo in conjunction with the assigned treatment in the 200 mg dose level group.

### Results:

COMPOUND is safe and well tolerated across the full range of single, oral doses from 1 to 200 mg. At doses≥10 mg, tₘₐₓ ranges from 1.3 to 3.0 h and terminal t_{1/2} from 17.8 to 23.6 h. The exposure increase across the dose range is essentially dose-proportional and there is no relevant food effect on the pharmacokinetics. COMPOUND was found in this study to be mainly excreted in feces and to a minor extent in urine. The absolute bioavailability was about 50%.

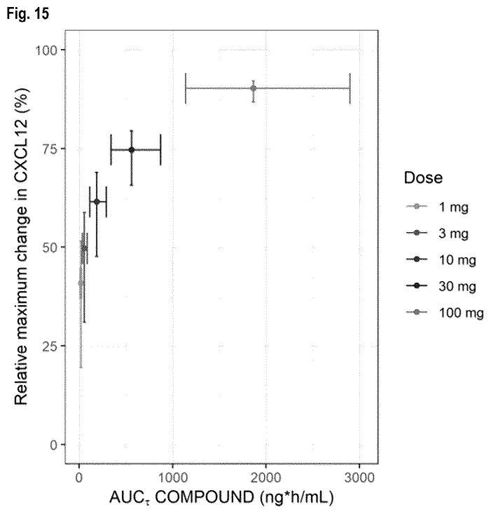

Plasma concentrations of the target engagement biomarker CXCL12 dose-dependently increase across the tested dose range with approximately 2-fold higher levels compared to baseline. Plasma CXC12 concentrations and fold changes compared to baseline are similar after administration of 100 mg and 200 mg COMPOUND (FIG. 14). An indirect-response pharmacokinetic/pharmacodynamic model predicting the exposure-response at steady state describes the relationship of COMPOUND and CXCL12 concentrations well (FIG. 15). In this study in healthy volunteers, CXCL11 concentrations remain essentially unchanged.

FIG. 14 shows the dose-response relationship of peak CXCL12 plasma concentrations in healthy subjects in the study. The data are presented as fold change compared to baseline with the horizontal lines representing the mean and dots representing individual data points.

FIG. 15 shows the predicted exposure response relationship at steady-state stratified by dose. The dots represent the median predicted fold change compared to baseline and error bars represent the 80% prediction interval.

### Example E: The Investigation of Safety, Tolerability, Pharmacokinetics and Pharmacodynamics of COMPOUND can be Determined after Multiple Doses in Healthy Male and Female Subjects

### A) Study Design (ClinicalTrials.Gov: NCT04286750)

In a randomized, double-blind, placebo-controlled study different dose levels of COMPOUND are investigated, for example 30, 100, and 200 mg. In each dose level group, eight healthy subjects (4 male and 4 female) receive COMPOUND and two healthy subjects (1 male and 1 female) receive matching placebo, orally, in the fasted condition once daily for 7 days, e.g. in the morning. During the 7 days of dosing and up to 8 days after the last dosing subjects are monitored to investigate (i) tolerability and safety (adverse events, vital signs, clinical laboratory, ECG), (ii) pharmacokinetics (COMPOUND concentration in plasma), and (iii) pharmacodynamics (including e.g. plasma levels of CXCL11 and CXCL12).

1. A pharmaceutical composition comprising, as active principles,
(3S,4S)-1-Cyclopropylmethyl-4-{[5-(2,4-difluoro-phenyl)-isoxazole-3-carbonyl]-amino}-piperidine-3-carboxylic acid (1-pyrimidin-2-yl-cyclopropyl)-amide: 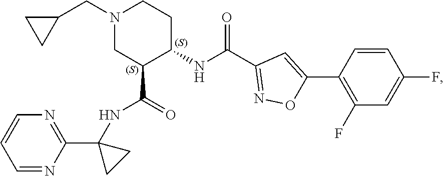
or a pharmaceutically acceptable salt thereof,
in combination with an S1P1 receptor modulator, or a pharmaceutically acceptable salt thereof,
as well as at least one pharmaceutically acceptable excipient.

2. The pharmaceutical composition according to claim 1, wherein said S1P1 receptor modulator is fingolimod, ponesimod, siponimod, ozanimod, cenerimod, or etrasimod; or a pharmaceutically acceptable salt thereof.

3. The pharmaceutical composition according to claim 1 or 2, wherein said S1P1 receptor modulator, or a pharmaceutically acceptable salt thereof, is comprised in a dose of said S1P1 receptor modulator which is a tolerated efficacious dose or lower than a tolerated efficacious dose of said S1P1 receptor modulator when given as a single therapy.

4. A method for prevention or treatment of an autoimmune or inflammatory disease or disorder, a transplant rejection, or a neurodegenerative disease or disorder, comprising administering (3S,4S)-1-Cyclopropylmethyl-4-{[5-(2,4-difluoro-phenyl)-isoxazole-3-carbonyl]-amino}-piperidine-3-carboxylic acid (1-pyrimidin-2-yl-cyclopropyl)-amide, or a pharmaceutically acceptable salt thereof, in combination with an S1P1 receptor modulator, or a pharmaceutically acceptable salt thereof.

5. The method of claim 4, wherein
the autoimmune and/or inflammatory disease and disorder is
an autoimmune and/or inflammatory demyelinating disease or disorder selected from multiple sclerosis (MS); idiopathic inflammatory demyelinating diseases; neuromyelitis optica spectrum diseases including neuromyelitis optica and acute optic neuritis; auto-immune encephalomyelitis including acute disseminated encephalomyelitis (ADEM) and multiphasic disseminated encephalomyelitis (MDEM); myelitis including transverse myelitis spectrum disorders, acute flaccid myelitis, poliomyelitis, leukomyelitis, and meningococcal myelitis; brain stem encephalitis; anti-myelin oligodendrocyte glycoprotein (anti-MOG) associated diseases including anti-MOG encephalomyelitis; Guillain-Barré syndrome; chronic inflammatory demyelinating polyneuropathy (CIDP); and anti-myelin-associated glycoprotein (anti-MAG) peripheral neuropathy;
rheumatoid arthritis (RA);
an inflammatory bowel disease (IBD) including Crohns disease or ulcerative colitis;
systemic lupus erythematosus (SLE) including lupus nephritis and neuropsychiatric systemic lupus erythematosus;
interstitial cystitis;
celiac disease;
osteoarthritis;
psoriasis;
type I diabetes;
ankylosing spondylitis; or
cytokine release syndrome following a strong viral infection or acute respiratory distress syndrome including COVID-19;
the transplant rejection is rejection of a transplanted organ such as kidney, liver, heart, lung, pancreas, cornea, or skin; graft-versus-host disease brought about by hematopoietic stem cell transplantation; chronic allograft rejection and chronic allograft vasculopathy; or
the neurodegenerative disease or disorder is amyotrophic lateral sclerosis (ALS), Huntington's disease, Alzheimer's disease (AD), Parkinson's disease (PD), or adrenoleukodystrophy.

6. The method of claim 5, wherein the method is for prevention or treatment of the autoimmune and/or inflammatory disease or disorder.

7. The method of claim 5, wherein said treatment reduces the rate of progression of said autoimmune and/or inflammatory disease or disorder.

8. The method of claim 7, wherein said autoimmune and/or inflammatory disease or disorder is
an autoimmune and/or inflammatory demyelinating disease or disorder selected from multiple sclerosis (MS); idiopathic inflammatory demyelinating diseases; auto-immune encephalomyelitis including acute disseminated encephalomyelitis (ADEM) and multiphasic disseminated encephalomyelitis (MDEM); myelitis including transverse myelitis spectrum disorders, acute flaccid myelitis, poliomyelitis, leukomyelitis, and meningococcal myelitis; brain stem encephalitis; Guillain-Barré syndrome; chronic inflammatory demyelinating polyneuropathy (CIDP); anti-myelin-associated glycoprotein (anti-MAG) peripheral neuropathy; and myelin oligodendrocyte glycoprotein (MOG)-antibody associated disease;
an inflammatory bowel disease especially selected from Crohn's disease and ulcerative colitis; or
systemic lupus erythematosus (SLE).

9. The method of claim 4, the method is for prevention or treatment of the autoimmune or inflammatory disease or disorder, and the autoimmune or inflammatory disease or disorder is an autoimmune and/or inflammatory demyelinating disease or disorder.

10. The method of claim 9, wherein
the method is for the treatment of a patient diagnosed with MS, wherein said treatment
reduces the rate of progression of MS; and/or
improves the symptoms of MS; and/or
reduces the rate of demyelination; and/or
reduces the rate of irreversible neurodegenerative damage such as axonal damage; and/or
has an effect of remyelination; and/or
reduces the rate of brain atrophy/cerebral atrophy; or
the method is for the prevention of MS, wherein said prevention of MS comprises delaying the onset of MS in a subject who is at risk/has been diagnosed as being at risk of developing MS; wherein said subject is especially a subject who has experienced a Clinically Isolated Syndrome (CIS) or has been diagnosed as having experienced a CIS.

11. The method of claim 4, wherein said S1P1 receptor modulator is fingolimod, ponesimod, siponimod, ozanimod, cenerimod, or etrasimod; or a pharmaceutically acceptable salt thereof.

12. The method of claim 11, wherein said S1P1 receptor modulator, or a pharmaceutically acceptable salt thereof, is administered in a pharmaceutical dosage form suitable for the oral administration of said S1P1 receptor modulator, wherein
fingolimod, or a pharmaceutically acceptable salt thereof, if present, is to be administered in said pharmaceutical dosage form in a unit dose suitable for the oral administration of a total of about 0.5 mg or below per day of fingolimod;
siponimod, or a pharmaceutically acceptable salt thereof, if present, is to be administered in said pharmaceutical dosage form in a unit dose suitable for the oral administration of a total of about 2 mg or below per day of siponimod;
ponesimod, or a pharmaceutically acceptable salt thereof, if present, is to be administered in said pharmaceutical dosage form in a unit dose suitable for the oral administration of a total of about 20 mg or below per day of ponesimod; and
ozanimod, or a pharmaceutically acceptable salt thereof, if present, is to be administered in said pharmaceutical dosage form in a unit dose suitable for the oral administration of a total of about 1 mg or below per day of ozanimod;
cenerimod, or a pharmaceutically acceptable salt thereof, if present, is to be administered in said pharmaceutical dosage form in a unit dose suitable for the oral administration of a total of about 4 mg or below per day of cenerimod; and
etrasimod, or a pharmaceutically acceptable salt thereof, if present, is to be administered in said pharmaceutical dosage form in a unit dose suitable for the oral administration of a total of about 2 mg or below per day of etrasimod.

13. The method of claim 4, wherein said S1P1 receptor modulator, or a pharmaceutically acceptable salt thereof, is to be administered in a dose which is a tolerated efficacious dose when given as a single therapy; or in a dose which is lower than such tolerated efficacious dose when given as a single therapy.

14. (canceled)

15. A method for the prophylaxis or treatment of an autoimmune or inflammatory disease or disorder, a transplant rejection, or a neurodegenerative disease or disorder; said method comprising the administration of a pharmaceutically effective amount of (3S,4S)-1-Cyclopropylmethyl-4-{[5-(2,4-difluoro-phenyl)-isoxazole-3-carbonyl]-amino}-piperidine-3-carboxylic acid (1-pyrimidin-2-yl-cyclopropyl)-amide, or of a pharmaceutically acceptable salt thereof, to a subject in need thereof, wherein the (3S,4S)-1-Cyclopropylmethyl-4-{[5-(2,4-difluoro-phenyl)-isoxazole-3-carbonyl]-amino}-piperidine-3-carboxylic acid (1-pyrimidin-2-yl-cyclopropyl)-amide is administered in combination with an S1P1 receptor modulator, or a pharmaceutically acceptable salt thereof.
